(19) United States
(12) Patent Application Publication
(10) Pub. No.: US 2015/0335681 A1
(43) Pub. Date: Nov. 26, 2015

(54) Biocomposites Having Viable Stem Cells and Platelets and Methods for Making and Using the Same

(71) Applicant: MicroAire Surgical Instruments, LLC, Charlottesville, VA (US)

(72) Inventor: John Raymond Chapman, Sacramento, CA (US)

(21) Appl. No.: 14/718,637

(22) Filed: May 21, 2015

Related U.S. Application Data
(60) Provisional application No. 62/002,052, filed on May 22, 2014.

(51) Int. Cl. A61K 35/19; A61L 27/54; A61L 27/36; A61L 27/38; A61K 35/28; A61K 45/06

(52) U.S. Cl. CPC A61K 35/19; A61K 35/28; A61K 45/06; A61L 27/3608; A61L 27/3886; A61L 27/54; A61L 2430/02

(57) ABSTRACT

Aspects of the present disclosure include methods for preparing biocomposites having viable stem cells and platelets. Methods according to certain embodiments include combining a composition that contains viable stem cells with a composition the contains platelets in a centrifugation vessel, subjecting the centrifugation vessel to a force of centrifugation to produce two or more fractions such that each fraction includes a component having different density and collecting a fraction from the centrifuge vessel that includes the platelets and viable stem cells. Liquid biocomposites having viable stem cells and platelets and methods for administering the biocomposites to a body site of a subject are also provided.

### CROSS-REFERENCE TO RELATED APPLICATIONS

Pursuant to 35 U.S.C. §119 (e), this application claims priority to the filing date of U.S. Provisional Patent Application No. 62/002,052, filed May 22, 2014, the disclosure of which is herein incorporated by reference.

### INTRODUCTION

Animals, including human beings, are susceptible to a barrage of events that lead to injuries to tissue, which injuries may require medical attention. For the most part, such wounds heal at a fairly steady and slow rate, being affected by many factors including the nature and site of the wound and the physiological state of the animal. When the serous or underlying vascularized layers of the human body are disrupted, the body mounts a complex inflammatory wound healing response to repair the injury. Initial trauma leads to a phase of acute inflammatory response. Body fluids containing plasma proteins, fibrin, antibodies and various blood cells flow into the wound. Scab formation takes place and inflammation occurs within a few hours along with the actions of neutrophils, monocytes and macrophages. Next, fibroplasia takes place causing an increase in wound tensile strength and stimulation of fibroblast proliferation and growth. Fibroblasts secrete collagen, a fibrous protein as part of connective tissue. Collagen deposition generally begins from the fifth day. Finally, maturational processes to close the wound proceed, with tensile strength of the wound increasing from crosslinking of collagen fibers and deposition of fibrous connective tissue to cause scar formation.

### SUMMARY

Aspects of the present disclosure include methods for preparing biocomposites having viable stem cells and platelets. Methods according to certain embodiments include combining a composition that contains viable stem cells with a composition the contains platelets in a centrifugation vessel, subjecting the centrifugation vessel to a force of centrifugation to produce two or more fractions such that each fraction includes a component having different density and collecting a fraction from the centrifuge vessel that includes the platelets and viable stem cells. Liquid biocomposites having viable stem cells and platelets and methods for administering the biocomposites to a body site of a subject are also provided.

A method is provided for the preparation of novel cell compositions having clinical utility. In embodiments, methods include combining a first composition containing viable stem cells and a second composition containing platelets in a centrifugation vessel. In some embodiments, the first composition is lipoaspirate. In some instances, the lipoaspirate includes viable stem cells and one or more of adipose tissue fragments, adipocytes, preadipocytes, fibroblasts, endothelial precursor cells, endothelial cells, macrophages, leukocytes and red blood cells. For example, the first composition may be a centrifuged fraction of lipoaspirate that includes mesenchymal stromal cells, red blood cells, macrophages, leukocytes and endothelial cells. In certain embodiments, the first composition is a fluid having mesenchymal stromal cells derived from adipose tissue and blood cells.

In some embodiments, the second composition is a blood composition, such as whole blood, peripheral blood or platelet-rich plasma. In other embodiments, the second composition is a non-blood composition that contains platelets such as bone marrow aspirate. In certain instances, the second composition is a centrifuged fraction of whole blood that includes platelets and red blood cells. In certain embodiments, the first composition and second composition are collected from the same subject. In one example, methods include admixing adipose and blood tissue derived cells and then performing a volume reduction and purification process to isolate a density fluid phase enriched in concentration for both blood derived platelets and adipose derived mesenchymal stromal cells.

In practicing the subject methods, the first composition and second composition are combined in a centrifugation vessel and subjected to a force of centrifugation sufficient to produce two or more fractions, where each fraction includes a component having a different density. In some embodiments, the centrifugation vessel is subjected to a centrifugal force of from 100 g to 10,000 g for a duration of from 1 minute to 120 minutes, such as a force of 1000 g for 60 minutes. In embodiments, a fraction that has a concentration of viable stems cells (e.g., mesenchymal stromal cells) and platelets that is 1.5-fold or greater than the first and second composition is collected from the centrifugation vessel. In some instances, the collected fraction having the viable stem cells and platelets has a volume of 30% or less of the combined first and second compositions. In certain embodiments, the fraction collected includes red blood cells, such as in an amount that is 50% or less than the red blood cells present in the first and second compositions.

In certain embodiments, methods include: 1) combining a composition having mesenchymal stromal cells derived from adipose tissue and blood cells with the fluid having platelets; 2) centrifuging to form density phase fractions; and 3) harvesting an intermediate density fraction containing the majority of platelets and mesenchymal stromal cells. In these embodiments, volume reduction, red cell depletion, and concentration of two cell populations derived from adipose and from blood which work synergistically in wound healing and achieving cosmetic effects when delivered to the body.

Aspects of the disclosure also include liquid biocomposites that contain a high concentration of viable stem cells and platelets. In embodiments, the liquid biocomposites have a concentration of viable stem cells of 1000 cells/mL or more and a platelet concentration of 1×10⁶ platelets/μL or more. In some embodiments, the biocomposites include adipose tissue fragments, adipocytes, preadipocytes, fibroblasts, endothelial precursor cells, endothelial cells, macrophages, leukocytes and red blood cells. In certain embodiments, the biocomposites are formulated for contacting with a body site of subject in the form of a gel, cream, foam or an aerosol. The biocomposites may also be combined with one or more components including fibrin, fibrinogen, plasmin, plasminogen and thrombin, such as for example, being incorporated into a fibrin gel. The biocomposites may also be combined with one or more bioactive agents. In certain instances, the biocomposites include a bone graft, such as bone chips or bone particles.

Aspects of the disclosure also include methods for administering one or more of the subject biocomposites containing a high concentration of viable stem cells and platelets to a body site of a subject. In some embodiments, the body site is a wound site (e.g., trauma or surgery). In some instances, the biocomposite is prepared and applied directly to the wound site, such as by applying the biocomposite in the form of a gel, cream, liquid or aerosol. In other instances, the biocomposite is combined with one or more other components, such as a fibrin gel or adipose tissue composite, in a container (e.g., in a three-dimensional mold) and applied to the body site. In certain embodiments, biocomposites of interest include one or more components that are derived from the subject's own body (i.e., one or more components are autologous).

In certain embodiments, the subject biocomposites facilitate delivery of therapeutic cells into a tissue space with limited volume capacity. Examples of tissues with limited space that require highly volume-reduced flood compartments include intra-tissue injections such as intra-muscular, intra-dermal, subcutaneous, intra-organ, and intra-thecal routes of administration. In these embodiments, methods include preparing a volume reduced mixed cell fraction having at least adipose derived nucleated cells and whole blood derived platelets. In other embodiments, the subject biocomposites are admixed cell compositions wherein the therapeutic potential of the cells contained have a synergistic effect providing a greater beneficial effect than if not admixed. For example, in certain embodiments, the biocomposites include growth factors which can signal the mesenchymal stem cells to provide a more rigorous wound healing response than if the growth factors were not present. In yet other embodiments, the subject methods reduce or avoid unnecessary delays and costs associated with the preparation of the therapeutic adipose derived and whole blood derived cell compositions. For example, this time and cost consideration can be important for point of care medical treatments such as those that occur in a physician's office, in a home setting for chronic wound care by a visiting health care worker or in the intra-operative theatre.

### BRIEF DESCRIPTION OF THE FIGURES

The invention may be best understood from the following detailed description when read in conjunction with the accompanying drawings. Included in the drawings are the following figures:

FIG. 1 is an operational flow chart for preparing a biocomposite according to certain embodiments.

FIG. 2 graphically depicts illustrative steps in the preparation of a biocomposite according to certain embodiments.

### DETAILED DESCRIPTION

Aspects of the present disclosure include methods for preparing biocomposites having viable stem cells and platelets. Methods according to certain embodiments include combining a composition that contains viable stem cells with a composition the contains platelets in a centrifugation vessel, subjecting the centrifugation vessel to a force of centrifugation to produce two or more fractions such that each fraction includes a component having different density and collecting a fraction from the centrifuge vessel that includes the platelets and viable stem cells. Liquid biocomposites having viable stem cells and platelets and methods for administering the biocomposites to a body site of a subject are also provided.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, the present disclosure provides methods for preparing biocomposites having viable stem cells and platelets. In further describing embodiments of the disclosure, methods for combining a first composition containing viable stem cells with a second composition containing platelets in a centrifugation vessel, subjecting the centrifugation vessel to a force of centrifugation and collecting one or more fractions from the centrifuge vessel are first described. Next, biocomposites having a high concentration of viable stems cells (e.g., mesenchymal stromal cells) and platelets are described. Methods for using the subject biocomposites as well as kits are also provided.

### Methods for Preparing Biocomposites Having Viable Stem Cells and Platelets

As summarized above, aspects of the disclosure include methods for preparing biocomposites having viable stem cells and platelets. In embodiments, methods include: 1) combining a first composition having viable stem cells with a second composition having platelets in a centrifugation vessel; 2) subjecting the centrifugation vessel to a force of centrifugation to produce two or more fractions such that each fraction includes a component having a different density; and 3) collecting a fraction from the centrifuge vessel that includes platelets and viable stem cells. As described in greater detail below, the subject biocomposites have a high concentration of viable stem cells and platelets as compared to the concentration of the viable stem cells in the first composition and the concentration of the platelets in the second composition. In other words, the subject methods provide a biocomposite of enriched viable stem cells and platelets in a significantly reduced volume. In certain embodiments, biocomposites containing enriched viable stem cells and platelets have a volume that is 50% or less of the combined volume of the first and second compositions, such as 45% or less, such as 40% or less, such as 30% or less, such as 20% or less and including 10% or less than the combined volume of the first and second compositions. Put another way, the concentration of viable stem cells and platelets in biocomposites of interest are increased as compared to the concentration of viable stem cells in the first composition and platelets in the second composition, such as by 10% or more, such as 25% or more, such as by 35% or more, such as by 50% or more, such as by 75% or more, such as by 90% or more and including by 99% or more. In certain embodiments, the concentration of viable stem cells and platelets in biocomposites of interest are each greater than the concentration of viable stem cells in the first composition and platelets in the second composition by 1.5-fold or more, such as 2-fold or more, such as 2.5-fold or more, such as 3-fold or more, such as 5-fold or more and including by 10-fold or more.

As described in greater detail below, the first composition includes viable stem cells and the second composition includes platelets. In embodiments, the first and second compositions may be biological samples, where the term “biological sample” is used in its conventional sense to include a whole organism, or a subset of animal tissues, cells or component parts which may in certain instances be found in blood, mucus, adipose tissue, bone marrow, lymphatic fluid, synovial fluid, cerebrospinal fluid, saliva, bronchoalveolar lavage, amniotic fluid, amniotic cord blood, urine, vaginal fluid and semen. As such, a “biological sample” refers to fluids and tissues from the organism as well as to a homogenate, lysate or extract prepared from the fluid or tissues, including but not limited to, for example, plasma, serum, adipose tissue fragments, spinal fluid, bone marrow, lymph fluid, sections of the skin, respiratory, gastrointestinal, cardiovascular, and genitourinary tracts, tears, saliva, milk, blood cells, tumors, organs. Biological samples may include any type of organismic material, including both healthy and diseased components (e.g., cancerous, malignant, necrotic, etc.). In certain embodiments, the biological sample is a liquid sample, such as whole blood or derivative thereof (e.g., plasma), tears, sweat, urine, semen, etc., where in some instances the sample is a blood sample, including whole blood, such as blood obtained from venipuncture or fingerstick (where the blood may or may not be combined with any reagents prior to assay, such as preservatives, anticoagulants, etc.).

The first composition includes at least one viable stem cell. The term “stem cells” is used herein in its conventional sense to refer to undifferentiated biological cells that can differentiate into specialized cells and can divide to produce more stem cells. In some embodiments, the first composition includes viable stem cells that are present in the lipoaspirate (e.g., adipose tissue), such as mesenchymal stem cells and stromal stem cells. In other embodiments, the first composition includes viable stem cells that have been added and may include purified hematopoietic and non-hematopoietic stem cells. By “viable” is meant that the stem cells are living and capable of maintaining or recovering the potentialities of stem cell activity (e.g., dividing, differentiating, etc.)

Depending on the source and volume of the first composition, the number of viable stem cells in the first composition may vary, such as 1 viable stem cell or greater, such as 10 viable stem cells or greater, such as 1×10² viable stem cells or greater, such as 5×10² viable stem cells or greater, such as 1×10³ viable stem cells or greater, such as 5×10³ viable stem cells or greater, such as 1×10⁴ viable stem cells or greater, such as 5×10⁴ viable stem cells or greater, such as 1×10⁵ viable stem cells or greater, such as 5×10⁵ viable stem cells or greater, such as 1×10⁶ viable stem cells or greater, such as 5×10⁶ viable stem cells or greater, such as 1×10⁷ viable stem cells or greater, such as 1×10⁸ viable stem cells or greater, such as 1×10⁹ viable stem cells or greater and including 1×10¹⁰ viable stem cells or greater. In some embodiments, the concentration of viable stem cells in the first composition is 10 viable stem cells/mL or more, 1×10² viable stem cells/mL or more, such as 5×10² viable stem cells/mL or more, such as 1×10³ viable stem cells/mL or more, such as 5×10³ viable stem cells/mL or more, such as 1×10⁴ viable stem cells/mL or more, such as 5×10⁴ viable stem cells/mL or more, such as 1×10⁵ viable stem cells/mL or more, such as 5×10⁵ viable stem cells/mL or more, such as 1×10⁶ viable stem cells/mL or more, such as 5×10⁶ viable stem cells/mL or more, such as 1×10⁷ viable stem cells/mL or more, such as 1×10⁸ viable stem cells/mL or more, such as 1×10⁹ viable stem cells/mL or more and including 1×10¹⁰ viable stem cells/mL or more.

In certain embodiments, the first composition also includes other nucleated cells, such as endothelial cells, macrophages, leukocytes and red blood cells or a combination thereof. When present, the concentration of each type of cell may vary ranging from 1×10² cells to 1×10¹⁰ cells, such as from 1×10³ cells to 1×10⁹ cells, such as from 1×10⁴ cells to 1×10⁸ cells and including from 1×10⁵ cells to 1×10⁷ cells. In one example, the first composition includes endothelial cells. In another example, the first composition includes macrophages. In still another example, the first composition includes leukocytes. In yet another example, the first composition includes red blood cells. Depending on the composition of the final biocomposite desired, the ratio of the number of viable stem cells to other nucleated cells in the first composition may vary, ranging from between 1:1 and 1:1.5; 1:1.5 and 1:2; 1:2 and 1:2.5; 1:2.5 and 1:3; 1:3 and 1:3.5; 1:3.5 and 1:4; 1:4 and 1:4.5; 1:4.5 and 1:5; 1:5 and 1:5.5; 1:5.5 and 1:6; 1:6 and 1:6.5; 1:6.5 and 1:7; 1:7 and 1:7.5; 1:7.5 and 1:8; 1:8 and 1:8.5; 1:8.5 and 1:9; 1:9 and 1:9.5; 1:9.5 and 1:10 or a range thereof. For example, the ratio of the number viable stem cells to other nucleated cells in the first composition may range from 1:1 and 1:10, such as 1:1 and 1:8, such as 1:1 and 1:5, such as 1:1 and 1:4, and including from 1:1 and 1:2. In certain instances, the ratio of the number of other nucleated cells to viable stem cells ranges from between 1:1 and 1:1.5; 1:1.5 and 1:2; 1:2 and 1:2.5; 1:2.5 and 1:3; 1:3 and 1:3.5; 1:3.5 and 1:4; 1:4 and 1:4.5; 1:4.5 and 1:5; 1:5 and 1:5.5; 1:5.5 and 1:6; 1:6 and 1:6.5; 1:6.5 and 1:7; 1:7 and 1:7.5; 1:7.5 and 1:8; 1:8 and 1:8.5; 1:8.5 and 1:9; 1:9 and 1:9.5; 1:9.5 and 1:10 or a range thereof. For example, the ratio of the number of other nucleated cells to viable stem cells may range from 1:1 and 1:10, such as 1:1 and 1:8, such as 1:1 and 1:5, such as 1:1 and 1:4, and including from 1:1 and 1:2.

In some embodiments, the first composition is lipoaspirate. In certain instances, the lipoaspirate includes viable stem cells (e.g., mesenchymal stromal cells), adipose tissue fragments, adipocytes, preadipocytes, fibroblasts, endothelial precursor cells, endothelial cells, macrophages, leukocytes and red blood cells. In these embodiments, methods may further include performing lipoplasty on a subject to harvest lipoaspirate. The term “lipoplasty” is used herein in its conventional sense to refer to the medical procedure for harvesting adipose tissue from a subject's body, such as by utilizing a cannula, suction source and a chamber to collect adipose tissue. In these embodiments, the lipoplasty may be carried out in such a way that the substantial majority of the cells in adipose tissue remain viable. Suitable lipoplasty protocols may include, but are not limited to suction assisted lipoplasty (SAL), ultra-sound assisted lipoplasty (USAL), power assisted lipoplasty (PAL), syringe assisted lipoplasty (SAL), laser assisted lipoplasty (LAL) and water jet assisted lipoplasty (WJAL). The adipose tissue harvested by lipoplasty contain excess liquid which can be removed by draining, by removal of supernatant after gentle centrifugation, by filtration or after spontaneous phase separation.

In practicing the subject methods, the amount of lipoaspirate harvested may depend on the desired volume of the first composition, ranging from 1 g to 10,000 g, such as from 5 g to 9000 g, such as from 10 g to 8000 g, such as from 15 g to 7000 g, such as from 20 g to 6000 g, such as from 25 g to 5000 g, such as from 30 g to 4000 g, such as from 35 g to 3000 g, such as from 40 g to 2000 g, such as from 50 g to 1000 g and including from 100 g to 500 g. As such, the volume of the first composition may be 1 mL or more, such as 2 mL or more, such as 5 mL or more, such as 10 mL or more, such as 25 mL or more, such as 50 mL or more, such as 100 mL or more, such as 250 mL or more, such as 500 mL or more, such as 750 mL or more and including 1000 mL or more. For example, the volume of the first composition contacted with the second composition may range from 1 mL to 1000 mL, such as from 5 mL to 900 mL, such as from 10 mL to 800 mL, such as from 15 mL to 700 mL, such as from 20 mL to 600 mL and including from 25 mL to 500 mL. When preparing the subject biocomposites, the lipoaspirate composition may be contacted directly with the second composition or may be further processed before combining with the second composition in the centrifugation vessel.

In some embodiments, the lipoaspirate is filtered. The lipoaspirate may be filtered by any convenient protocol, including but not limited to using gravity or vacuum filtration through filter paper or a ceramic frit. In certain instances, the lipoaspirate is filtered by conveying the lipoaspirate through a mesh screen, such as a 2 mesh screen or smaller, such as a 4 mesh screen or smaller, such as a 10 mesh screen or smaller, such as a 20 mesh screen or smaller, such as a 30 mesh screen or smaller, such as a 40 mesh screen or smaller, and including a 60 mesh screen or smaller.

In other embodiments, the lipoaspirate is subjected to a force of centrifugation sufficient to separate the lipoaspirate into two or more fractions having components of different density. In these embodiments, one or more of the fractions may be collected and used as the first composition. In certain instances, the lipoaspirate is separated into two or more fractions by centrifugation in a lipoaspirate processing apparatus, such as described in U.S. Patent Publication No. 2013/0210600, filed Feb. 15, 2013, the disclosure of which is herein incorporated by reference. For example, one or more of the fractions of the centrifuged lipoaspirate may be combined with the second composition to prepare biocomposites containing a high concentration of viable stem cells and platelets.

In embodiments, the second composition includes platelets. In some instances, the second composition is a blood sample. The term “blood sample” refers to whole blood or a subset of blood components, including but not limited to platelets, red blood cells, white cells and blood plasma. In some embodiments, the blood sample is obtained from an in vivo source and can include blood samples obtained from tissues (e.g., cell suspension from a tissue biopsy, cell suspension from a tissue sample, etc.) or directly from a subject. In some cases, blood samples derived from a subject are cultured, stored, or manipulated prior to evaluation. In one example, the blood sample is whole blood. In another example, the blood sample is peripheral blood. In another example, the blood sample is platelet rich plasma. In other embodiments, the second composition is a non-blood sample that contains platelets, such as bone marrow aspirate.

Depending on the source and volume of the second composition, the number of platelets in the second composition may vary, such as 1×10⁴ platelets or greater, such as 5×10⁴ platelets or greater, such as 1×10⁵ platelets or greater, such as 5×10⁵ platelets or greater, such as 1×10⁶ platelets or greater, such as 5×10⁶ platelets or greater, such as 1×10⁷ platelets or greater, such as 5×10⁷ platelets or greater, such as 1×10⁸ platelets or greater, such as 1×10⁹ platelets or greater, such as 1×10¹⁰ platelets or greater, such as 1×10¹² platelets or greater, such as 1×10¹⁴ platelets or greater and including 1×10¹⁶ platelets or greater. In some embodiments, the concentration of platelets in the first composition is 1×10⁴ platelets/μL or more, such as 5×10⁴ platelets/μL or more, such as 1×10⁵ platelets/μL or more, such as 5×10⁵ platelets/μL or more, such as 1×10⁶ platelets/μL or more, such as 5×10⁶ platelets/μL or more, such as 1×10⁷ platelets/μL or more, such as 5×10⁷ platelets/μL or more, such as 1×10⁸ platelets/μL or more, such as 5×10⁸ platelets/μL or more, such as 1×10⁹ platelets/μL or more, such as 1×10⁹ platelets/μL or more, such as 1×10¹⁰ platelets/μL or more and including 1×10¹⁵ platelets/μL or more.

In certain embodiments, the second composition also includes other components, such as leukocytes and red blood cells or a combination thereof. Where desired, the concentration of leukocytes and red blood cells in the second composition may vary ranging from 1×10² cells to 1×10¹⁰ cells, such as from 1×10³ cells to 1×10⁹ cells, such as from 1×10⁴ cells to 1×10⁸ cells and including from 1×10⁵ cells to 1×10⁷ cells. In one example, the second composition includes leukocytes. In another example, the second composition includes red blood cells. In still another example, the second composition includes leukocytes and red blood cells. Depending on the composition of the final biocomposite desired, the ratio of the number of platelets to leukocytes and red blood cells in the second composition may vary, ranging from between 1:1 and 1:1.5; 1:1.5 and 1:2; 1:2 and 1:2.5; 1:2.5 and 1:3; 1:3 and 1:3.5; 1:3.5 and 1:4; 1:4 and 1:4.5; 1:4.5 and 1:5; 1:5 and 1:5.5; 1:5.5 and 1:6; 1:6 and 1:6.5; 1:6.5 and 1:7; 1:7 and 1:7.5; 1:7.5 and 1:8; 1:8 and 1:8.5; 1:8.5 and 1:9; 1:9 and 1:9.5; 1:9.5 and 1:10 or a range thereof. For example, the ratio of the number of platelets to other leukocytes and red blood cells in the second composition may range from 1:1 and 1:10, such as 1:1 and 1:8, such as 1:1 and 1:5, such as 1:1 and 1:4, and including from 1:1 and 1:2. In certain instances, the ratio of leukocytes and red blood cells to platelets ranges from between 1:1 and 1:1.5; 1:1.5 and 1:2; 1:2 and 1:2.5; 1:2.5 and 1:3; 1:3 and 1:3.5; 1:3.5 and 1:4; 1:4 and 1:4.5; 1:4.5 and 1:5; 1:5 and 1:5.5; 1:5.5 and 1:6; 1:6 and 1:6.5; 1:6.5 and 1:7; 1:7 and 1:7.5; 1:7.5 and 1:8; 1:8 and 1:8.5; 1:8.5 and 1:9; 1:9 and 1:9.5; 1:9.5 and 1:10 or a range thereof. For example, the ratio of leukocytes and red blood cells to platelets may range from 1:1 and 1:10, such as 1:1 and 1:8, such as 1:1 and 1:5, such as 1:1 and 1:4, and including from 1:1 and 1:2.

In some embodiments, the second composition is whole blood. In other embodiments, the second composition is peripheral blood. In still other embodiments, the second composition is bone marrow aspirate. In certain embodiments, the second composition is a derivative portion of a biological sample, such as a composition where the concentration of platelets in the biological sample has been increased, for example by centrifugation and fractionation. For instance, the second composition may be a derivative of a biological sample where the concentration of platelets has been increased by 5% or more, such as by 10% or more, such as by 20% or more, such as by 25% or more, such as by 30% or more, such as by 50% or more, such as by 75% or more, such as 90% including by 95% or more as compared to the biological sample that is not subjected to centrifugation and fractionation. In certain embodiments, the second composition is a derivative of a biological sample where the concentration of platelets has been increased by 2-fold or more, such as by 3-fold or more, such as by 5-fold or more, such as by 7-fold or more and including by 10-fold or more. For example, in some instances the second composition is whole blood that has been subjected to a force of centrifugation and fractionated such that the second composition includes 15% or less of the volume of plasma of the whole blood sample, such as 10% or less, such as 8% or less and including 5% or less of the volume of plasma of the whole blood sample. In certain embodiments, the second composition is whole blood that has been subjected to a force of centrifugation and fractionated such that the second composition includes 15% or less of the red blood cells of the whole blood sample, such as 10% or less, such as 8% or less and including 5% or less of the red blood cells of the whole blood sample. In still other embodiments, the second composition is whole blood that has been subjected to a force of centrifugation and fractionated such that the second composition includes 15% or less of the leukocytes of the whole blood sample, such as 10% or less, such as 8% or less and including 5% or less of the leukocytes of the whole blood sample.

In certain embodiments, the second composition is platelet-rich plasma such as a composition obtained by centrifugation and fractionation as described in co-pending U.S. patent application Ser. No. 13/199,129 filed on Aug. 19, 2011, U.S. patent application Ser. No. 13/199,111 filed on Aug. 19, 2011, U.S. patent application Ser. No. 13/199,119 filed on Aug. 19, 2011 as well as U.S. Provisional Patent Application No. 62/069,783 filed on Oct. 28, 2014, the disclosures of which are herein incorporated by reference.

In practicing the subject methods, the volume of the second composition containing platelets may be 1 mL or more, such as 2 mL or more, such as 5 mL or more, such as 10 mL or more, such as 25 mL or more, such as 50 mL or more, such as 100 mL or more, such as 250 mL or more, such as 500 mL or more, such as 750 mL or more and including 1000 mL or more. For example, the volume of the second composition may range from 1 mL to 1000 mL, such as from 5 mL to 900 mL, such as from 10 mL to 800 mL, such as from 15 mL to 700 mL, such as from 20 mL to 600 mL and including from 25 mL to 500 mL.

In practicing the subject methods, the composition containing viable stem cells and the platelet composition is combined in a centrifugation vessel and subjected to a force of centrifugation. Any convenient centrifugation vessel may be employed so long as the applied force of centrifugation is sufficient to produce two or more fractions having components of different density in the centrifugation vessel. In certain embodiments, the centrifugation vessel contains a floating buoy such as described in U.S. Provisional Patent Application No. 62/069,783 filed on Oct. 28, 2014, the disclosure of which is herein incorporated by reference.

The term “force of centrifugation” is used herein in its conventional sense to refer to the force applied to the sample in the centrifugation vessel (i.e., the combined first and second compositions) through revolving the device about an axis of rotation where the force on the components of the sample is in certain embodiments, given by the relative centrifugal force (RCF). The force of centrifugation may be applied by any convenient protocol, where in certain embodiments, the force of centrifugation is applied by a centrifuge. In these embodiments, any convenient centrifuge may be employed, such as for example a fixed-angle centrifuge, a swinging bucket centrifuge, ultracentrifuge, solid bowl centrifuges, conical centrifuges, among other types of centrifuges. As described in greater detail below, the applied force of centrifugation (in relative centrifugal force, RCF) may vary depending on the sample type and size and may range from 1 g to 50,000 g, such as from 2 g to 45,000 g, such as from 3 g to 40,000 g, such as from 5 g to 35,000 g, such as from 10 g to 25,000 g, such as from 100 g to 20,000 g, such as from 500 g to 15,000 g and including from 100 g to 10,000 g. In certain embodiments, the applied force of centrifugation is 1000 g.

In some embodiments, the combined first and second composition in the centrifugation vessel is subjected to the centrifugation force immediately after being introduced into the centrifugation vessel. In other embodiments, the combined first and second composition is subjected to the centrifugation force a predetermined period of time after being introduced into the centrifugation vessel. For example, the combined first and second composition may be subjected to the centrifugation force 0.01 minutes or more after being introduced into the device container, such as after 0.05 minutes or more, such as after 0.1 minutes or more, such as after 0.5 minutes or more, such as after 1 minute or more, such as after 5 minutes or more, such as after 10 minutes or more, such as after 15 minutes or more, such as after 30 minutes or more and including 60 minutes after being introduced into the centrifugation vessel.

In certain embodiments, methods include a storage or prefabrication step where the combined first and second composition is preloaded into the centrifugation vessel and stored for a predetermined period of time before being subjected to the centrifugation force. The amount of time the combined first and second composition is preloaded and stored in the centrifugation vessel may vary, such as 0.1 hours or more, such as 0.5 hours or more, such as 1 hour or more, such as 2 hours or more, such as 4 hours or more, such as 8 hours or more, such as 16 hours or more, such as 24 hours or more, such as 48 hours or more, such as 72 hours or more, such as 96 hours or more, such as 120 hours or more, such as 144 hours or more, such as 168 hours or more and including preloading for 240 hours or more. For example, the amount of time the combined first and second composition is preloaded and stored may range from 0.1 hours to 240 hours, such as from 0.5 hours to 216 hours, such as from 1 hour to 192 hours and including preloading the sample from 5 hours to 168 hours before being subjected to the centrifugation force. For instance, the combined first and second composition may be preloaded into the centrifugation vessel at a remote location (e.g., using in a physician's office or outpatient clinic) and sent to a laboratory for processing in accordance with the subject methods. By “remote location” is meant a location other than the location at which either the first composition and second composition are obtained and preloaded. For example, a remote location could be another location (e.g. office, lab, etc.) in the same city, another location in a different city, another location in a different state, another location in a different country, etc., relative to the location of the processing device, e.g., as described in greater detail below. In some instances, two locations are remote from one another if they are separated from each other by a distance of 10 m or more, such as 50 m or more, including 100 m or more, e.g., 500 m or more, 1000 m or more, 10,000 m or more, etc.

In embodiments, the combined first and second composition is subjected to a force of centrifugation for a duration sufficient to separate components of different density into two or more fractions. The duration the combined first and second composition is subjected to the force of centrifugation may vary and may be 0.01 minutes or longer, such as for 0.05 minutes or longer, such as for 0.1 minutes or longer, such as for 0.5 minutes or longer, such as for 1 minute or longer, such as for 3 minutes or longer, such as for 5 minutes or longer, such as for 10 minutes or longer, such as for 15 minutes or longer, such as for 20 minutes or longer, such as for 30 minutes or longer, such as for 45 minutes or longer, such as for 60 minutes or longer and including for 90 minutes or longer. For example, the combined first and second composition may be subjected to force of centrifugation for a duration which ranges from 0.01 minutes to 960 minutes, such as from 0.05 minutes to 480 minutes, such as from 0.1 minutes to 240 minutes, such as from 0.5 minutes to 120 minutes, such as from 1 minute to 90 minutes, such as from 2 minutes to 60 minutes and including from 10 minutes to 45 minutes.

Depending on the volume and density dispersity of the sample components, the rotational speed of centrifugation may vary, such as from 1×10³ revolutions per minute (rpm) to 1000×10³ rpm, such as from 2×10³ rpm to 900×10³ rpm, such as from 3×10³ rpm to 800×10³ rpm, such as from 4×10³ rpm to 700×10³ rpm, such as from 5×10³ rpm to 600×10³ rpm, such as from 10×10³ rpm to 500×10³ rpm and including from 25×10³ rpm to 100×10³ rpm. The centrifuge may be maintained at a single speed or may be changed to a different speed at any time during separation. Where the centrifuge is operated at more than one speed, the duration the centrifuge is maintained at each speed may independently be 0.01 minutes or more, such as 0.1 minutes or more, such as 1 minute or more, such as 5 minutes or more, such as 10 minutes or more, such as 30 minutes or more and including 60 minutes or more. The time period between each different speed employed may also vary, as desired, being separated independently by a delay of 1 minute or more, such as 5 minutes or more, such as by 10 minutes or more, such as by 15 minutes or more, such as by 30 minutes or more and including by 60 minutes or more. In embodiments where the centrifuge is maintained at more than two (i.e., three or more) speed to subject the combined first and second composition to the centrifugation force, the delay between each speed employed may be the same or different.

The centrifuge may be operated to apply the centrifugation force continuously or in discrete intervals. For example, in some embodiments, the centrifuge is operated to apply the centrifugation force continuously. In other instances, the centrifuge is operated to apply a centrifugation force in discrete intervals, such as for example for intervals of for 0.01 minutes or longer, such as for 0.05 minutes or longer, such as for 0.1 minutes or longer, such as for 0.5 minutes or longer, such as for 1 minute or longer, such as for 3 minutes or longer, such as for 5 minutes or longer, such as for 10 minutes or longer, such as for 15 minutes or longer, such as for 20 minutes or longer, such as for 30 minutes or longer, such as for 45 minutes or longer, such as for 60 minutes or longer and including for 90 minutes or longer. Where the centrifuge is operated to apply the force of centrifugation in discrete intervals, methods may include 1 or more intervals, such as 2 or more intervals, such as 3 or more intervals and including 5 or more intervals. In certain embodiments, methods include applying the force of centrifugation only one time. In other words, methods according to this embodiment are characterized by a single application of the centrifugation force to the combined first and second composition, such as by centrifuging the centrifugation vessel for a single spin interval.

Each step of the subject methods (combining the first composition and the second composition in the centrifugation vessel, subjecting the centrifugation vessel to a centrifugation force and collecting one or more of the separated fractions) can be carried out at any suitable temperature so long as the viability of the components (e.g., stem cells, platelets, red blood cells, leukocytes, platelets, etc.) are preserved as desired. As such, the temperature according to embodiments of the disclosure may vary, such as from −80° C. to 100° C., such as from −75° C. to 75° C., such as from −50° C. to 50° C., such as from −25° C. to 25° C., such as from −10° C. to 10° C., and including from 0° C. to 25° C.

Where necessary, the parameters for applying the force of centrifugation may be changed at any time during methods of the present disclosure. For example, the speed and duration of centrifugation and heating or cooling may be changed one or more times during the subject methods, such as two or more times, such as three or more times and including five or more times.

In some embodiments, methods include changing the speed of the centrifuge, such as by increasing or decreasing the speed by 1% or more, such as by 5% or more, such as by 10% or more, such as by 25% or more, such as by 50% or more, such as by 75% or more, such as by 90% or more, such as by 2-fold or more, such as by 5-fold or more, such as by 10-fold or more and including by 25-fold or more. For example, the speed of the centrifuge may be increased or decreased by 0.5×10³ rpm or more, such as by 1×10³ rpm or more, such as by 2×10³ rpm or more, such as by 5×10³ rpm or more, such as by 10×10³ rpm or more, such as by 25×10³ rpm or more and including increasing or decreasing the speed of the centrifuge by 100×10³ rpm or more.

In other embodiments, the duration of centrifugation may be changed. For example, the duration may be increased or decrease by 0.01 minutes or longer, such as by 0.05 minutes or longer, such as by 0.1 minutes or longer, such as by 0.5 minutes or longer, such as by 1 minute or longer, such as by 3 minutes or longer, such as by 5 minutes or longer, such as by 10 minutes or longer, such as by 15 minutes or longer, such as by 20 minutes or longer, such as by 30 minutes or longer, such as by 45 minutes or longer, such as by 60 minutes or longer and including by 90 minutes or longer.

In yet other embodiments, the temperature during centrifugation may be changed. For example, the temperature may be raised or lower by 0.1° C. or more, such as by 0.5° C. or more, such as by 1° C. or more, such as by 2° C. or more, such as by 5° C. or more and including raising or lowering the temperature by 8° C. or more.

In certain embodiments, methods include monitoring the centrifuged sample. Monitoring may include assessing (either by a human or with the assistance of a computer, if using a computer-automated process initially set up under human direction) the extent of component separation within the sample. For example, monitoring separation of components by density into the two or more fractions within the sample may include visually determining fraction boundaries between components of the sample. Monitoring separation of components may also include assessing the physical and chemical properties of the components in each fraction within the sample. Any convenient protocol can be employed to monitor the sample, including but not limited to visual observation, laser scatter, fluorescence, phosphorescence, chemiluminescence, diffuse reflectance, infrared spectroscopy, among other sensing protocols.

In some instances, monitoring includes collecting real-time data, such as employing a detector (e.g., with a video camera). In other instances, monitoring includes assessing the sample at regular intervals, such as every 0.01 minutes, every 0.05 minutes, every 0.1 minutes, every 0.5 minutes, every 1 minute, every 5 minutes, every 10 minutes, every 30 minutes, every 60 minutes or some other interval.

Methods of the present disclosure may also include a step of assessing the sample to identify any desired adjustments to the subject protocol. In other words, methods in these embodiments include providing feedback based on monitoring the sample, where adjustments to the protocol may vary in terms of goal, where in some instances the desired adjustment are adjustments that ultimately result in an improved fractionation of components by density within the sample, such as providing faster separation, improved purity or increased component enrichment (e.g., viable stem cells and platelets).

Where feedback provided indicates that a particular protocol is less than optimal, such as where separation requires too much time or where separation provides separated fractions with insufficient enrichment (e.g., viable stem cells and platelets are not sufficiently concentrated into a single fraction), methods may include changing one or more parts of the subject protocols. For example, one or more parameters of centrifugation may be adjusted. In one example, methods include adjusting the speed of the centrifuge (as described above). In another example, methods include changing (increasing or decreasing) the duration of centrifugation. In yet another example, methods include heating or cooling.

Depending on the components in the first and second compositions (as described above), the force of centrifugation is sufficient to produce two or more fractions where each fraction contains a component of different density. In some embodiments, the applied force of centrifugation is sufficient to produce three fractions: 1) an intermediate density fraction containing viable stem cells (e.g., mesenchymal stromal cells) and platelets; 2) a cell-free low density fluid fraction; and 3) a high density packed red blood cell fraction. In other embodiments, the applied force of centrifugation is sufficient to produce three fractions: 1) an intermediate density fraction containing viable stem cells (e.g., mesenchymal stromal cells), platelets, endothelial cells, macrophages and leukocytes; 2) a cell-free low density fluid fraction; and 3) a high density packed red blood cell fraction. In still other embodiments, the applied force of centrifugation is sufficient to produce three fractions: 1) an intermediate density fraction containing viable stem cells (e.g., mesenchymal stromal cells), platelets, endothelial cells, macrophages, leukocytes, adipocytes and fibroblasts; 2) a cell-free low density fluid fraction; and 3) a high density packed red blood cell fraction.

In embodiments of the present disclosure, methods include collecting the intermediate density fraction containing viable stem cells and platelets (and where present, endothelial cells, macrophages, leukocytes, adipocytes and fibroblasts). This fraction may be collected using any suitable collecting protocol, such as aspirating using a syringe with or without a needle, a manual or mechanically operated serological pipette as well as with an automated liquid collection system (e.g., a computer-controlled collection apparatus). Where the first and second compositions are combined in two or more centrifugation vessels, collecting fractions may include combining fractions of similar makeup. For example, where the first and second compositions are combined in two or more centrifugation vessels, the intermediate density fraction containing viable stem cells, platelets and where present endothelial cells, macrophages, leukocytes, adipocytes and fibroblasts are collected from each of the centrifugation vessels and combined.

The fraction containing viable stem cells and platelets may be collected at any time after subjecting the centrifugation vessel to the force of centrifugation. In some embodiments, the desired fraction is collected 1 minute or greater after the separated fractions are prepared, such as 2 minutes or greater, such as 3 minutes or greater, such as 5 minutes or greater, such as 10 minutes or greater and including 30 minutes or greater after the desired fraction is prepared.

In certain embodiments, a portion of the intermediate density fraction is collected, such as 5% or more, such as 10% or more, such as 25% or more, such as 50% or more, such as 75% or more and including 90% or more. In certain instances, the entire intermediate density fraction that contains viable stem cells and platelets is collected. To collect a portion of the intermediate density fraction, a liquid collection device (e.g., needle with syringe) may be positioned a predetermined depth into the intermediate density fraction, such as 1 mm or more into the intermediate density fraction, such as 2 mm or more, such as 3 mm or more, such as 5 mm or more, such as 10 mm or more and including 25 mm or more into the intermediate density fraction. In certain embodiments, the liquid collection device is positioned to a depth as determined by one or more reference indicators on the centrifugation vessel. In still other embodiments, the liquid collection device is positioned to a depth relative to the bottom boundary of the intermediate density fraction proximal end, such as 1 mm or more above the bottom boundary of the intermediate density fraction, such as 2 mm or more, such as 3 mm or more, such as 5 mm or more, such as 10 mm or more and including 25 mm or more above the bottom boundary of the intermediate density fraction.

In some embodiments, methods include removing a portion of the high density red blood cell fraction along with the intermediate density fraction containing viable stem cells and platelets. For example, 1% or more of the high density red blood cell fraction may be collected along with the intermediate density fraction containing viable stem cells and platelets, such as 2% or more, such as 5% or more, such as 10% or more and including 25% or more. In certain embodiments, 50% or less of the red blood cell fraction is collected, such as 45% or less, such as 40% or less, such as 35% or less, such as 25% or less, such as 15% or less, such as 10% or less and including 5% or less.

The high density red blood cell fraction may be collected concurrently with the intermediate density fraction containing viable stem cells and platelets or may be collected discrete before or after collecting the intermediate density fraction containing viable stem cells and platelets. In one example, a portion of the high density red blood cell fraction is collected before collecting the intermediate density fraction containing viable stem cells and platelets. In another example, a portion of the high density red blood cell fraction is collected after collecting the intermediate density fraction containing viable stem cells and platelets. In still another example, a portion of the high density red blood cell fraction is collected concurrently while collecting the intermediate density fraction containing viable stem cells and platelets. To collect a portion of the high density red blood cell fraction, a liquid collection device (e.g., needle with syringe) may be positioned a predetermined depth into the high density red blood cell fraction, such as 1 mm or more into the red blood cell fraction, such as 2 mm or more, such as 3 mm or more, such as 5 mm or more, such as 10 mm or more and including 25 mm or more into the red blood cell fraction. In certain embodiments, the liquid collection device is positioned to a depth as determined by one or more reference indicators on the centrifugation vessel. In still other embodiments, the liquid collection device is positioned to a depth relative to the bottom of the centrifugation vessel, such as 1 mm or more above the bottom of the centrifugation vessel, such as 2 mm or more, such as 3 mm or more, such as 5 mm or more, such as 10 mm or more and including 25 mm or more above the bottom of the centrifugation vessel.

As discussed above, the intermediate density fraction collected is a biocomposite that contains a high concentration of viable stem cells and platelets as compared to the concentration of viable stem cells in the first composition and platelets in the second composition. For example, in embodiments, the collected biocomposite has a concentration of viable stem cells and platelets that are individually 10% or more than that present in the first and second compositions, such as 25% or more, such as by 35% or more, such as by 50% or more, such as by 75% or more, such as by 90% or more and including by 99% or more. In certain embodiments, the concentration of viable stem cells and platelets in the collected fraction of interest is greater than the concentration of viable stem cells in the first composition and platelets in the second composition by 1.5-fold or more, such as 2-fold or more, such as 2.5-fold or more, such as 3-fold or more, such as 5-fold or more and including by 10-fold or more.

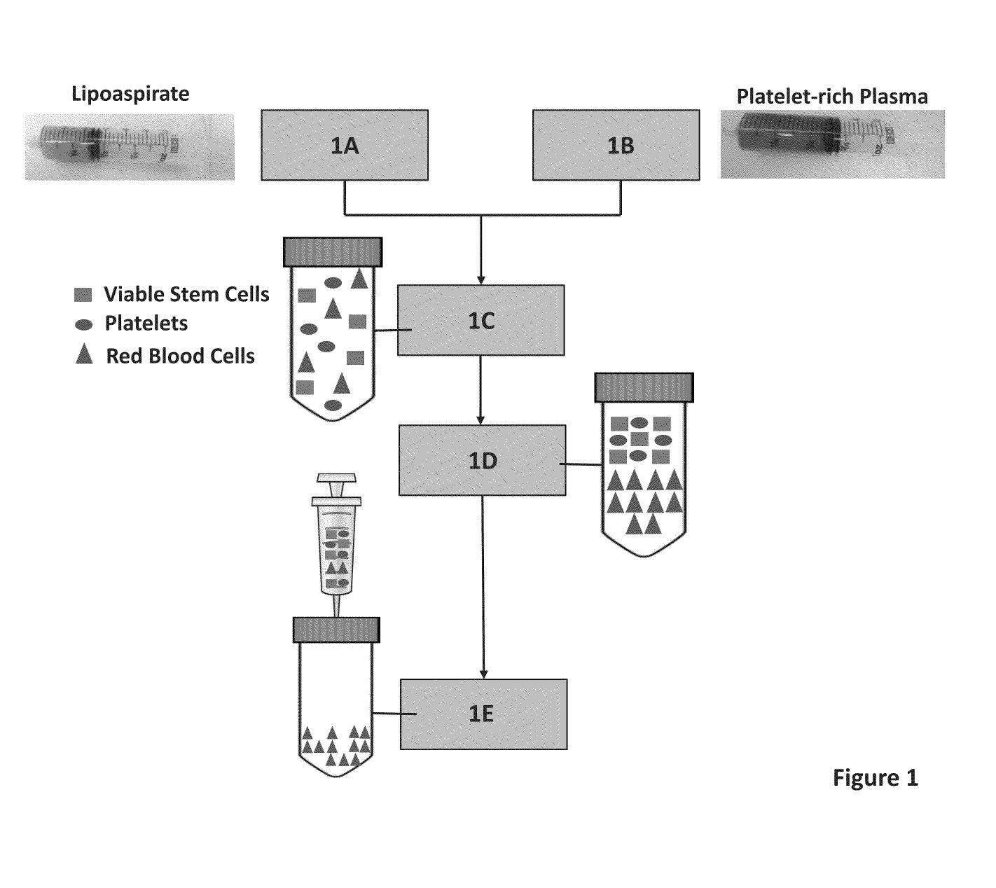

FIG. 1 illustrates step-by-step methods for preparing a biocomposite that contains a high concentration of viable stem cells and platelets according to certain embodiments. At step 1A, a sample of lipoaspirate fluid is harvested from the body by lipoplasty. The lipoaspirate contains adipose tissue fragments, oil released from lysed adipocytes and an aqueous fluid phase containing saline with various cells including viable stem cells (e.g., mesenchymal stromal cells) and red blood cells. A fraction of the aqueous supernatant can be discarded and the cells at the bottom of the collected lipoaspirate are re-suspended and include red blood cells and viable stem cells (e.g., mesenchymal stromal cells) as well as other nucleated cells, such as leukocytes and endothelial cells. At step 1B, whole blood is collected by phlebotomy, such as whole blood that is anticoagulated with citrate phosphate dextrose (CPD) and citrate dextrose (ACD) anticoagulants. At step 1C, the next step of the method is to combine the blood or fraction thereof that contains platelets and the lipoaspirate or fraction thereof that contains viable stem cells (e.g., mesenchymal stromal cells). In this embodiment, the combined sample has a composition having cellular constituents of varying density. The mixing may be performed in any suitable vessel for retaining the combined volume of the two fluids. In some embodiments of the present invention, it is useful that the vessel used for mixing the two fluids is the centrifugation vessel. At step 1D, a force of centrifugation is applied to the combined compositions and is of sufficient acceleration and time to separate the mixture into at least three phases: a low density phase substantially depleted of cells (i.e., cell-free low density fluidic fraction), an intermediate density phase containing platelets and viable stem cells (e.g., mesenchymal stromal cells) as well as other nucleated cells derived from blood or lipoaspirate (e.g., leukocytes, macrophages, endothelial cells, adipocytes) and a high density phase that includes packed red blood cells. As desired, it may be sufficient to only partially separate the sample into differing density fractions, or a greater degree of completeness of separation may be pursued, such as by operating the centrifuge for a longer period of time or changing the geometry of the centrifuge. At step 1E, once the applied force of centrifugation (i.e., centrifuge spinning) is stopped the centrifuge vessel is removed from the centrifuge and placed on a bench top. The intermediate density fraction that contains the highly concentrated viable stem cells and platelets can be harvested by pulling back the plunger of a syringe connected by tubing means to the location of the intermediate density fraction containing the majority of platelets and viable stem cells.

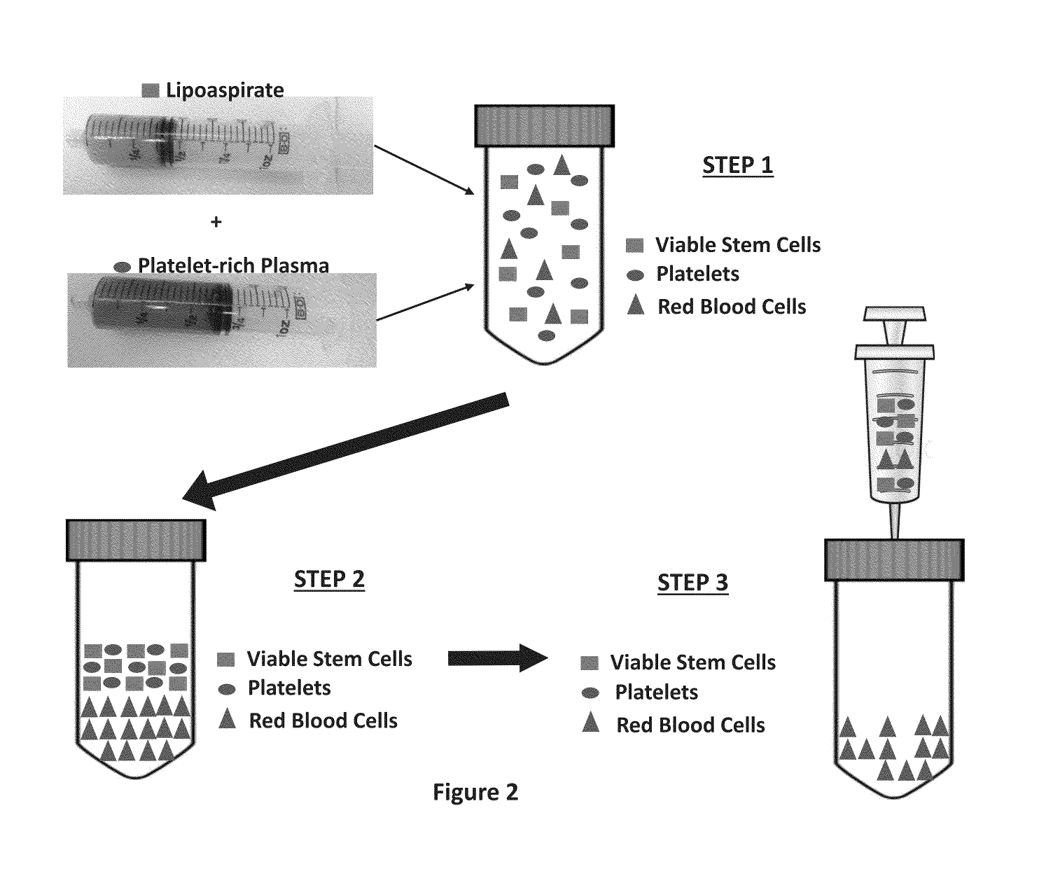

FIG. 2 graphically illustrates methods for preparing a biocomposite containing a high concentration of viable stem cells and platelets according to certain embodiments. At step 1, a first composition containing viable stem cells and a second composition containing platelets and red blood cells are combined in a centrifugation vessel. Centrifugation at step 2 provides stratified fractions having components of different densities. The upper fraction includes a cell-free low-density liquid phase. The bottom fraction is a high density packed red blood cell fraction. The intermediate fraction (positioned between the low density liquid phase and high density packed red blood cell phase) contains the viable stem cells and platelets. The intermediate density fraction containing viable stem cells and platelets is collected in step 3 using a syringe. In certain instances, a portion of the red blood cells present in the bottom high density fraction is collected with the intermediate density fraction containing viable stem cells and platelets.

### Biocomposites Containing Viable Stem Cells and Platelets

As summarized above, aspects of the present disclosure also include biocomposites containing viable stem cells and platelets. In embodiments, the biocomposites are liquid compositions that contain a high concentration of both viable stem cells and platelets, such as a concentration of 1000 viable stem cells/mL or more and a concentration of 1×10⁶ platelets/μL or more. As discussed in greater detail below, the subject biocomposites containing viable stem cells and platelets may be configured to be applied to a body site of a subject, such as a wound site. In describing biocomposites of interest, the term “subject” is meant the person or organism to which the biocomposite is applied and maintained in contact. As such, subjects may include but are not limited to mammals, e.g., humans and other primates, such as chimpanzees and other apes and monkey species; and the like, as well as non-human subjects such as, but not limited to, birds, mice, rats, dogs, cats, livestock and horses. In certain embodiments, the subject is a human.

The biocomposites containing viable stem cells and platelets may be configured to be applied to any convenient internal or external location on the subject, such as to organ tissue including but not limited to integumentary tissue (e.g. sections of the skin), oral tissue (e.g., buccal, tongue, palatal, gums), respiratory tissue (e.g., pharynx, larynx, trachea, bronchi, lungs, diaphragm) gastrointestinal tissue (e.g., esophagus, stomach, liver, gallbladder, pancreas, intestines, colon, rectum and anus.), cardiovascular tissue (e.g., heart, blood vessels), endocrine tissue (e.g., hypothalamus, pituitary gland, pineal body or pineal gland, thyroid, parathyroids, adrenal glands) and genitourinary tissue (kidneys, ureters, bladder, urethra, ovaries, fallopian tubes, uterus, vagina, mammary glands, testes, vas deferens, seminal vesicles, prostate, penis), muscular tissue, nervous tissue (e.g., brain, spinal cord, nerves) as well as soft skeletal tissue (cartilage, ligaments, tendons). Furthermore, the biocomposites may be applied to any type of organismic tissue, including both healthy and diseased tissue (e.g., cancerous, malignant, necrotic, etc.), where desired.

Biocomposites of interest contain a high concentration of viable stem cells and platelets. Depending on the volume of the biocomposite, the number of viable stem cells is 1×10² viable stem cells or greater, such as 5×10² viable stem cells or greater, such as 1×10³ viable stem cells or greater, such as 5×10³ viable stem cells or greater, such as 1×10⁴ viable stem cells or greater, such as 5×10⁴ viable stem cells or greater, such as 1×10⁵ viable stem cells or greater, such as 5×10⁵ viable stem cells or greater, such as 1×10⁶ viable stem cells or greater, such as 5×10⁶ viable stem cells or greater, such as 1×10⁷ viable stem cells or greater, such as 1×10⁸ viable stem cells or greater, such as 1×10⁹ viable stem cells or greater and including 1×10¹⁰ viable stem cells or greater. In embodiments of the present disclosure, the concentration of viable stem cells is 1×10³ viable stem cells/mL or more, such as 5×10³ viable stem cells/mL or more, such as 1×10⁴ viable stem cells/mL or more, such as 5×10⁴ viable stem cells/mL or more, such as 1×10⁵ viable stem cells/mL or more, such as 5×10⁵ viable stem cells/mL or more, such as 1×10⁶ viable stem cells/mL or more, such as 5×10⁶ viable stem cells/mL or more, such as 1×10⁷ viable stem cells/mL or more, such as 1×10⁸ viable stem cells/mL or more, such as 1×10⁹ viable stem cells/mL or more and including 1×10¹⁰ viable stem cells/mL or more. As discussed above, the term “stem cell” is used herein in its conventional sense to refer to undifferentiated biological cells that can differentiate into specialized cells and can divide to produce more stem cells. In some embodiments, the first composition includes viable stem cells that are present in the lipoaspirate (e.g., adipose tissue), such as mesenchymal stem cells and stromal stem cells. In other embodiments, biocomposite includes viable stem cells that have been added and may include purified hematopoietic and non-hematopoietic stem cells. By “viable” is meant that the stem cells are living and capable of maintaining or recovering the potentialities of stem cell activity (e.g., dividing, differentiating, etc.)

The amount of platelets in the subject biocomposites may vary, and may be 1×10⁴ platelets or greater, such as 5×10⁴ platelets or greater, such as 1×10⁵ platelets or greater, such as 5×10⁵ platelets or greater, such as 1×10⁶ platelets or greater, such as 5×10⁶ platelets or greater, such as 1×10⁷ platelets or greater, such as 5×10⁷ platelets or greater, such as 1×10⁸ platelets or greater, such as 1×10⁹ platelets or greater, such as 1×10¹⁰ platelets or greater, such as 1×10¹² platelets or greater, such as 1×10¹⁴ platelets or greater and including 1×10¹⁶ platelets or greater. In embodiments of the present disclosure, the concentration of platelets is 1×10⁶ platelets/μL or more, such as 5×10⁶ platelets/μL or more, such as 1×10⁷ platelets/μL or more, such as 5×10⁷ platelets/μL or more, such as 1×10⁸ platelets/μL or more, such as 5×10⁸ platelets/μL or more, such as 1×10⁹ platelets/μL or more, such as 1×10⁹ platelets/μL or more, such as 1×10¹⁰ platelets/μL or more and including 1×10¹⁶ platelets/μL or more.

Depending on the desired properties of the biocomposite and the type of tissue being applied (as discussed in greater detail below), the ratio of viable stem cells and platelets may vary, ranging from between 1:1 and 1:1.5; 1:1.5 and 1:2; 1:2 and 1:2.5; 1:2.5 and 1:3; 1:3 and 1:3.5; 1:3.5 and 1:4; 1:4 and 1:4.5; 1:4.5 and 1:5; 1:5 and 1:5.5; 1:5.5 and 1:6; 1:6 and 1:6.5; 1:6.5 and 1:7; 1:7 and 1:7.5; 1:7.5 and 1:8; 1:8 and 1:8.5; 1:8.5 and 1:9; 1:9 and 1:9.5; 1:9.5 and 1:10 or a range thereof. For example, the ratio of viable stem cells to platelets may range from 1:1 and 1:10, such as 1:1 and 1:8, such as 1:1 and 1:5, such as 1:1 and 1:4, and including from 1:1 and 1:2. In other embodiments, the ratio of platelets to viable stem cells ranges from between 1:1 and 1:1.5; 1:1.5 and 1:2; 1:2 and 1:2.5; 1:2.5 and 1:3; 1:3 and 1:3.5; 1:3.5 and 1:4; 1:4 and 1:4.5; 1:4.5 and 1:5; 1:5 and 1:5.5; 1:5.5 and 1:6; 1:6 and 1:6.5; 1:6.5 and 1:7; 1:7 and 1:7.5; 1:7.5 and 1:8; 1:8 and 1:8.5; 1:8.5 and 1:9; 1:9 and 1:9.5; 1:9.5 and 1:10 or a range thereof. For example, the ratio of platelets to viable stem cells may range from 1:1 and 1:10, such as 1:1 and 1:8, such as 1:1 and 1:5, such as 1:1 and 1:4, and including from 1:1 and 1:2.

In certain embodiments, biocomposites of interest also include other components, such as other nucleated cells found in lipoaspirate and whole blood, including but not limited to red blood cells, leukocytes, macrophages, adipocytes, preadipocytes, fibroblasts, endothelial precursor cells and endothelial cells or any combination thereof. When present, the amount of other nucleated cells found in the subject biocomposites varies, ranging from 1×10² cells to 1×10¹⁰ cells, such as from 1×10³ cells to 1×10⁹ cells, such as from 1×10⁴ cells to 1×10⁸ cells and including from 1×10⁵ cells to 1×10⁷ cells. For example, each type of cell may be present at a concentration of 1×10³ cells/mL or more, such as 5×10³ cells/mL or more, such as 1×10⁴ cells/mL or more, such as 5×10⁴ cells/mL or more, such as 1×10⁵ cells/mL or more, such as 5×10⁵ cells/mL or more, such as 1×10⁶ cells/mL or more, such as 5×10⁶ cells/mL or more, such as 1×10⁷ cells/mL or more, such as 1×10⁸ cells/mL or more, such as 1×10⁹ cells/mL or more and including 1×10¹⁰ cells/mL or more. For example, the concentration of leukocytes and red blood cells may range from 1×10² cells to 1×10¹⁰ cells, such as from 1×10³ cells to 1×10⁹ cells, such as from 1×10⁴ cells to 1×10⁸ cells and including from 1×10⁵ cells to 1×10⁷ cells.

In some embodiments, the subject biocomposites further include adipose tissue, such as in the form of adipose tissue fragments. The term “adipose tissue” is used herein in its conventional sense to refer to the lipophilic connective tissue in the body that contains adipocytes as well as the stromal vascular fraction having preadipocytes, fibroblasts, vascular endothelial cells, mesenchymal stem cells, immune cells (e.g., adipose tissue macrophages) as well as endothelial precursor cells that can secrete tissue repair proteins (e.g., tissue plasminogen activator, tissue plasminogen inhibitor). Adipose tissue, in some instances, also includes the hormones produced by the adipose tissue, such as leptin, estrogen, resistin, and the cytokine TNFα. In some embodiments, adipose tissue is fat obtained from the body of the subject, such as abdominal fat, epicardial fat, subcutaneous fat and ectopic fat, among other types of fats.

In some embodiments, the adipose tissue is in the form of a plurality of adipose tissue fragments. The fragments may be homogeneous in shape and size or more may be different. In some embodiments, the adipose tissue fragments have the same shapes and sizes. In other embodiments, the adipose tissue fragments have different shapes and sizes. The size of the fragments vary depending on the source of the adipose tissue as well as any processing following obtaining the adipose tissue fragments and may have a median diameter which ranges, such as from 1 μm to 5000 μm, such as from 10 μm to 4500 μm, such as from 50 μm to 4000 μm, such as from 75 μm to 3500 μm, such as from 100 μm to 3000 μm, such as from 250 μm to 2500 μm and including a median diameter from 500 μm to 1500 μm. The amount of adipose tissue in the adipose tissue biocomposites may vary ranging from 1 g to 10,000 g, such as from 5 g to 9000 g, such as from 10 g to 8000 g, such as from 15 g to 7000 g, such as from 20 g to 6000 g, such as from 25 g to 5000 g, such as from 30 g to 4000 g, such as from 35 g to 3000 g, such as from 40 g to 2000 g, such as from 50 g to 1000 g and including from 100 g to 500 g.

In certain embodiments, the adipose tissue also includes tissue plasminogen activator. Tissue plasminogen activator refers to the serine protease protein that catalyzes the conversion of plasminogen to plasmin. In some embodiments, tissue plasminogen activator is native to the adipose tissue or has been later added to the adipose tissue, such as in the form of purified tissue plasminogen activator or recombinant tissue plasminogen activator. The amount of tissue plasminogen activator in the adipose tissue may vary, ranging from 0.01 μg to 100 μg, such as from 0.05 μg to 90 μg, such as from 0.1 μg to 80 μg, such as from 0.5 μg to 70 μg, such as from 1 μg to 60 μg and including from 5 μg to 50 μg. In these embodiments, the expressed activity of the tissue plasminogen activator may range from 1×10² IU/mg to 1×10⁸ IU/mg, such as from 5×10² IU/mg to 5×10⁷ IU/mg, such as from 1×10³ IU/mg to 1×10⁷ IU/mg, such as from 5×10³ IU/mg to 5×10⁶ IU/mg and including from 1×10⁴ IU/mg to 1×10⁶ IU/mg.

Adipose tissue may be taken from lipoaspirate, such as described above. For instance, the adipose tissue may be taken from lipoaspirate obtained from a subject by a lipoplasty protocol such as suction assisted lipoplasty (SAL), ultra-sound assisted lipoplasty (USAL), power assisted lipoplasty (PAL), syringe assisted lipoplasty (SAL), laser assisted lipoplasty (LAL) or water jet assisted lipoplasty (WJAL), among other lipoplasty protocols.

The amount of adipose tissue in the subject biocomposites may vary, ranging from 1 g to 10,000 g, such as from 5 g to 9000 g, such as from 10 g to 8000 g, such as from 15 g to 7000 g, such as from 20 g to 6000 g, such as from 25 g to 5000 g, such as from 30 g to 4000 g, such as from 35 g to 3000 g, such as from 40 g to 2000 g, such as from 50 g to 1000 g and including from 100 g to 500 g.

In certain embodiments, biocomposites containing viable stem cells and platelets are combined with a fibrin network. By “network” is meant a crosslinked array of polymerized fibrin. The term “crosslinked” is used its conventional sense to refer to the physical (e.g., intermolecular interactions or entanglements, such as through hydrophobic interactions) or chemical (e.g., covalent bonding) interaction between backbone components of polymer precursors. In some embodiments, the fibrin network includes polymerized strands of fibrin. In other embodiments, the fibrin network includes polymerized strands of fibrin and platelets. In yet other embodiments, the fibrin network includes polymerized strands of fibrin that are crosslinked by Factor XIII.

In certain embodiments, the fibrin network is prepared by combining thrombin and fibrinogen with the biocomposite containing viable stem cells and platelets. The amount of thrombin may vary depending on the desired mechanical and tensile strength and malleability of the subject adipose tissue biocomposite and may include 0.01 μg to 100 μg, such as from 0.05 μg to 90 μg, such as from 0.1 μg to 80 μg, such as from 0.5 μg to 70 μg, such as from 1 μg to 60 μg and including from 5 μg to 50 μg. In these embodiments, the expressed activity of the thrombin in preparing the subject biocomposites may range from 1×10² IU/mg to 1×10⁸IU/mg, such as from 5×10² IU/mg to 5×10⁷ IU/mg, such as from 1×10³ IU/mg to 1×10⁷ IU/mg, such as from 5×10³ IU/mg to 5×10⁶ IU/mg and including from 1×10⁴ IU/mg to 1×10⁶ IU/mg.

In these embodiments, the fibrinogen and thrombin may be present in a fluid composition together, such as in whole blood, autologous whole blood plasma anti-coagulated with a calcium-chelating agent, platelet rich plasma with its associated growth factors, autologous plasma, autologous platelet rich plasma, plasma and collagen mixture as represented by Vitagel by Orthovita; purified allogeneic fibrinogen with added thrombin. In certain embodiments, the fibrinogen is present in plasma (e.g., platelet rich plasma, platelet-poor plasma), such as plasma obtained prepared using a centrifugation vessel such as described in co-pending U.S. patent application Ser. No. 13/199,129 filed on Aug. 19, 2011, U.S. patent application Ser. No. 13/199,111 filed on Aug. 19, 2011, U.S. patent application Ser. No. 13/199,119 filed on Aug. 19, 2011 as well as U.S. Provisional Patent Application No. 62/069,783 filed on Oct. 28, 2014, the disclosures of which are herein incorporated by reference. In some embodiments, thrombin is prepared such as described in International Patent Application No. PCT/US2013/061756 published as WO2014/052496 on Apr. 3, 2014, the disclosure of which is herein incorporated by reference.

The amount of fibrin may vary depending on the amount of fibrinogen combined with thrombin the in subject biocomposites and may range from 1 g to 1000 g, such as from 5 g to 900 g, such as from 10 g to 800 g, such as from 15 g to 700 g, such as from 20 g to 600 g, such as from 25 g to 500 g, such as from 30 g to 400 g, such as from 35 g to 300 g, such as from 40 g to 200 g and including from 50 g to 100 g.

In certain embodiments, the fibrin network includes plasminogen. In some instances, the fibrin network includes plasminogen that is present in one or more of the thrombin source and fibrinogen source. In other instances, the fibrin network includes plasminogen added to one or more of the thrombin source and fibrinogen source, such as purified plasminogen as well as recombinant plasminogen. The amount of plasminogen in the fibrin network may vary, ranging from 0.01 μg to 100 μg, such as from 0.05 μg to 90 μg, such as from 0.1 μg to 80 μg, such as from 0.5 μg to 70 μg, such as from 1 μg to 60 μg and including from 5 μg to 50 μg. In these embodiments, the expressed activity of plasminogen may range from 1×10² IU/mg to 1×10⁸IU/mg, such as from 5×10² IU/mg to 5×10⁷ IU/mg, such as from 1×10³ IU/mg to 1×10⁷ IU/mg, such as from 5×10³ IU/mg to 5×10⁶ IU/mg and including from 1×10⁴ IU/mg to 1×10⁶ IU/mg.

In other embodiments, the fibrin network includes plasmin. In some instances, the fibrin network includes plasmin that is present in one or more of the thrombin source and fibrinogen source. In other instances, the fibrin network includes plasmin added to one or more of the thrombin source and fibrinogen source, such as purified plasmin as well as recombinant plasmin. The amount of plasmin in the fibrin network may vary, ranging from 0.01 μg to 100 μg, such as from 0.05 μg to 90 μg, such as from 0.1 μg to 80 μg, such as from 0.5 μg to 70 μg, such as from 1 μg to 60 μg and including from 5 μg to 50 μg. In these embodiments, the expressed activity of plasmin may range from 1×10² IU/mg to 1×10⁸ IU/mg, such as from 5×10² IU/mg to 5×10⁷ IU/mg, such as from 1×10³ IU/mg to 1×10⁷ IU/mg, such as from 5×10³ IU/mg to 5×10⁶ IU/mg and including from 1×10⁴ IU/mg to 1×10⁶ IU/mg.

When the subject biocomposites containing viable stem cells and platelets are combined with thrombin and fibrinogen to produce a fibrin network, the fibrin network may have a crosslink density which ranges from 1×10⁻¹⁵ moles/cm³ to 1×10⁻³ moles/cm³, such as 1×10⁻¹⁴ moles/cm³ to 1×10⁻³ moles/cm³, such as 1×10⁻¹³ moles/cm³ to 1×10⁻³ moles/cm³, such as 1×10⁻¹² moles/cm³ to 1×10⁻³ moles/cm³, such as 1×10⁻¹¹ moles/cm³ to 1×10⁻³ moles/cm³, such as 1×10⁻¹⁰ moles/cm³ to 1×10⁻³ moles/cm³, such as 1×10⁻⁹ moles/cm³ to 1×10⁻³ moles/cm³, such as 1×10⁻⁸ moles/cm³ to 1×10⁻³ moles/cm³, such as 1×10⁻¹¹ moles/cm³ to 1×10⁻⁷ moles/cm³, and including 1×10⁻⁶ moles/cm³ to 1×10⁻³ moles/cm³. Likewise, the compressive modulus may vary, ranging from 1 kPa to 35 kPa, such as from 2 kPa to 33 kPa, such as from 3 kPa to 30 kPa, such as from 4 kPa to 28 kPa, such as form 5 kPa to 25 kPa, such as from 6 kPa to 22 kPa, such as from 7 kPa to 20 kPa and including a compressive modulus ranging from 10 kPa to 20 kPa.

In embodiments, the source of thrombin may be any convenient source including, but not limited to, autologous thrombin serum, autologous thrombin serum supplemented with ethanol, allogeneic thrombin serum, allogeneic thrombin serum supplemented with ethanol, bovine thrombin, recombinant thrombin, and human thrombin derived from pooled plasma. In some embodiments, thrombin is from whole blood. In other embodiments, thrombin is from plasma. The source of fibrinogen, plasminogen and plasmin may also vary, as desired, including by not limited to autologous whole blood anti-coagulated with a calcium-chelating agent, platelet rich plasma with its associated growth factors, autologous plasma, autologous platelet rich plasma, plasma and collagen mixture as represented by Vitagel by Orthovita; purified allogeneic fibrinogen as represented by Tisseel/Tissucol and Beriplast products or by Quixil® consisting of a cross-linked allogeneic fibrinogen-fibronectin multimers and other naturally occurring adhesive glycoproteins to promote adhesion to collagen. In some embodiments, one or more of fibrinogen, plasminogen and plasmin are from whole blood. In other embodiments, one or more of fibrinogen, plasminogen and plasmin are from plasma. As discussed in greater detail below, in certain instances, the subject adipose tissue biocomposites are prepared in a body site (e.g., would site) and the fibrin network is prepared from thrombin, fibrinogen, plasminogen and plasmin present at the body site.

As discussed in greater detail below, in some embodiments the subject biocomposites containing viable stem cells and platelets may be applied directly to a tissue of a subject. In other embodiments, where the biocomposites are further prepared with one or more of a fibrin network (i.e., combining with a source of fibrinogen and thrombin) and adipose tissue, the subject biocomposites may be first prepared as a castable composition. The term “castable” is used in its conventional sense to refer to a composition that can be molded into a desired shape (e.g., by placing the composition into a shaped mold or body cavity) and may be subsequently hardened to form the final desired biocomposite. As such, the subject biocomposites may be formed into any convenient shape and size, such as a planar shape, including a circle, oval, half-circle, crescent-shaped, star-shaped, square, triangle, rhomboid, pentagon, hexagon, heptagon, octagon, rectangle or other suitable polygon or a three-dimensional shape, such as in the shape of a cube, cone, half sphere, star, triangular prism, rectangular prism, hexagonal prism or other suitable polyhedron as well as in the shape of thin tubes.

Where the biocomposite is cast into a planar shape, the surface area may range from 0.1 to 100 cm², such as 0.5 to 75 cm², such as 1.0 to 50 cm², such as 1.5 to 45 cm², such as 2.0 to 40 cm², such as 2.5 to 35 cm², and including 2 to 30 cm². Where the biocomposite is cast into a three-dimensional shape, the size may range from 0.1 to 100 cm³, such as 0.5 to 75 cm³, such as 1.0 to 50 cm³, such as 1.5 to 45 cm³, such as 2.0 to 40 cm³, such as 2.5 to 35 cm³, and including 2 to 30 cm³. The thickness of the biocomposite may be 0.1 mm or more, such as 0.5 mm or more, such as 1 mm or more, such as 2 mm or more, such as 3 mm or more, such as 5 mm or more, such as 10 mm or more, such as 25 mm or more, such as 50 mm or more and including 100 mm or more. For example, the overall thickness of the biocomposite may range from 1 mm to 100 mm, such as from 2 mm to 90 mm, such as from 3 mm to 75 mm and including a thickness of from 5 mm to 50 mm. In some embodiments, biocomposites are configured into one or more layers, such as two or more layers, such as three or more layers, such as 4 or more layers and including 5 or more layers.

In some embodiments, the subject biocomposites having viable stem cells and platelets may include one or more bioactive agents, such as two or more types of bioactive agents, such as three or more types of bioactive agents, such as four or more types of bioactive agents, such as five or more types of bioactive agents and including ten or more types of bioactive agents.

Example bioactive agents according to embodiments of the disclosure may include but are not limited to interferon, interleukin, erythropoietin, granulocyte-colony stimulating factor (GCSF), stem cell factor (SCI:), leptin (OB protein), interferon (alpha, beta, gamma), antibiotics such as vancomycin, gentamicin ciprofloxacin, amoxycillin, lactobacillus, cefotaxime, levofloxacin, cefipime, mebendazole, ampicillin, lactobacillus, cloxacillin, norfloxacin, tinidazole, cefpodoxime, proxctil, azithromycin, gatifloxacin, roxithromycin, cephalosporin, anti-thrombogenics, aspirin, ticlopidine, sulfinpyrazone, heparin, warfarin, growth factors, differentiation factors, hepatocyte stimulating factor, plasmacytoma growth factor, glial derived neurotrophic factor (GDNF), neurotrophic factor 3 (NT3), fibroblast growth factor (FGF), transforming growth factor (TGF), platelet transforming growth factor, milk growth factor, endothelial growth factors, endothelial cell-derived growth factors (ECDGF), alpha-endothelial growth factors, beta-endothelial growth factor, neurotrophic growth factor, nerve growth factor (NGF), vascular endothelial growth factor (VEGF), 4-1 BB receptor (4-IBBR), TRAIL (TNF-related apoptosis inducing ligand), artemin (GFRalpha3-RET ligand), BCA-I (B cell-attracting chemokinel), B lymphocyte chemoattractant (BLC), B cell maturation protein (BCMA), brain-derived neurotrophic factor (BDNF), bone growth factor such as osteoprotegerin (OPG), bone-derived growth factor, thrombopoietin, megakaryocyte derived growth factor (MDGF), keratinocyte growth factor (KGF), platelet-derived growth factor (PDGF), ciliary neurotrophic factor (CNTF), neurotrophin 4 (NT4), granulocyte colony-stimulating factor (GCSF), macrophage colony-stimulating factor (mCSF), bone morphogenetic protein 2 (BMP2), BRAK, C-IO, Cardiotrophin 1 (CTI), CCR8, anti-inflammatory: paracetamol, salsalate, diflunisal, mefenamic acid, diclofenac, piroxicam, ketoprofen, dipyrone, acetylsalicylic acid, anti-cancer drugs such as aliteretinoin, altertamine, anastrozole, azathioprine, bicalutarnide, busulfan, capecitabine, carboplatin, cisplatin, cyclophosphamide, cytarabine, doxorubicin, epirubicin, etoposide, exemestane, vincristine, vinorelbine, hormones, thyroid stimulating hormone (TSH), sex hormone binding globulin (SHBG), prolactin, luteotropic hormone (LTH), lactogenic hormone, parathyroid hormone (PTH), melanin concentrating hormone (MCH), luteinizing hormone (LHb), growth hormone (HGH), follicle stimulating hormone (FSHb), haloperidol, indomethacin, doxorubicin, epirubicin, amphotericin B, Taxol, cyclophosphamide, cisplatin, methotrexate, pyrene, amphotericin B, anti-dyskinesia agents, Alzheimer vaccine, antiparkinson agents, ions, edetic acid, nutrients, glucocorticoids, heparin, anticoagulation agents, antivirus agents, anti-HIV agents, polyamine, histamine and derivatives thereof, cystineamine and derivatives thereof, diphenhydramine and derivatives, orphenadrine and derivatives, muscarinic antagonist, phenoxybenzamine and derivatives thereof, protein A, streptavidin, amino acid, beta-galactosidase, methylene blue, protein kinases, beta-amyloid, lipopolysaccharides, eukaryotic initiation factor-4G, tumor necrosis factor (TNF), tumor necrosis factor-binding protein (TNF-bp), interleukin-1 (to 18) receptor antagonist (IL-Ira), granulocyte macrophage colony stimulating factor (GM-CSF), novel erythropoiesis stimulating protein (NESP), thrombopoietin, tissue plasminogen activator (TPA), urokinase, streptokinase, kallikrein, insulin, steroid, acetaminophen, analgesics, antitumor preparations, anti-cancer preparations, anti-proliferative preparations or pro-apoptotic preparations, among other types of bioactive agents.

The amount of each bioactive agent may vary depending on the type of bioactive agent and may be 0.01 μg or more, such as 0.05 μg or more, such as 0.1 μg or more, such as 0.5 μg or more, such as 1 μg or more, such as 5 μg or more, such as 10 μg or more, such as 25 μg or more, such as 50 μg or more, such as 100 μg or more, such as 250 μg or more, such as 1000 μg or more, such as 10 g or more, such as 25 g or more and including 100 g of bioactive agent or more. Where the bioactive agent is a liquid, the concentration of each bioactive agent may be 0.0001 μg/mL or greater, such as 0.001 μg/mL or greater, such as 0.01 μg/mL or greater, such as 0.1 μg/mL or greater, such as 0.5 μg/mL or greater, such as 1 μg/mL or greater, such as 2 μg/mL or greater, such as 5 μg/mL or greater, such as 10 μg/mL or greater, such as 25 μg/mL or greater, such as 50 μg/mL or greater, such as 100 μg/mL or greater such as 500 μg/mL or greater, such as 1 g/mL or greater such as 5 g/mL or greater and including 10 g/mL or greater.

Where more than one bioactive agent combined with the subject biocomposites, the amount (i.e., mass) of each of bioactive agent may vary, ranging from 0.001 mg to 1000 mg, such as 0.01 mg to 500 mg, such as 0.1 mg to 250 mg, such as 0.5 mg to 100 mg, such as 1 mg to 50 mg, including 1 mg to 10 mg. As such, in compositions of the invention, the mass ratio of the first bioactive agent to other (i.e., second or more) bioactive agent may vary, and in some instances may range between 1:1 and 1:2.5; 1:2.5 and 1:5; 1:5 and 1:10; 1:10 and 1:25; 1:25 and 1:50; 1:50 and 1:100; 1:100 and 1:150; 1:150 and 1:200; 1:200 and 1:250; 1:250 and 1:500; 1:500 and 1:1000, or a range thereof. For example, the mass ratio of the first bioactive agent to other (i.e., second or more) bioactive agents may range between 1:1 and 1:10; 1:5 and 1:25; 1:10 and 1:50; 1:25 and 1:100; 1:50 and 1:500; or 1:100 and 1:1000.

Depending on any additive components combined with the subject biocomposites containing viable stem cells and platelets, the biocomposite may be in the form of a liquid solution or suspension, syrup, gel, foam or formulated for aerosolized administration.

### Methods for Applying Biocomposites Containing Viable Stem Cells and Platelets to a Subject

As summarized above, the subject disclosure provides biocomposites that contain a high concentration of viable stem cells and platelets. Aspects of the disclosure also include methods for applying one or more of the subject biocomposites to a subject. Methods of using the subject biocomposites include applying one or more of the biocomposites to a body site of the subject in order to treat a subject for a target condition of interest such as for wound repair (in trauma wounds or surgical wounds). By “treating” or “treatment” is meant at least a suppression or amelioration of the symptoms associated with the condition affecting the subject, where suppression and amelioration are used in a broad sense to refer to at least a reduction in the magnitude of a parameter, e.g., symptom, associated with the condition being treated. As such, treatment also includes situations where the condition is completely inhibited, e.g., prevented from happening, or stopped, e.g., terminated, such that the subject no longer experiences the condition. As such, treatment includes both preventing and managing a condition.

In certain embodiments, biocomposites containing viable stem cells and platelets as described herein are used in the treatment of a wound site, such as where the wound occurs by injury or by surgery. In other embodiments, the subject biocomposites are used to separate tissues during tissue repair (e.g., remesothelialization) such as during healing from a wound or to prevent the formation of tissue adhesions. In other embodiments, the subject biocomposites are used to prevent or treat fistulas. In still other embodiments, the subject biocomposites are used to reduce time for wound closure, reduce scarring from a healed wound, to treat a skin excision, to treat skin ulcers, to treat tissue burns (e.g., skin burns) or to deliver one or more bioactive agents to the body site, such as by sustained or pulsatile release, as described above. In one example, the subject biocomposites are used to promote hemostasis. For instance, the biocomposite may be combined with a bone graft (e.g., bone chips, cadaver, synthetic or autologous) configured as an osteohemostatic plug.

In embodiments, methods include applying one or more of the subject biocomposites to a body site of a subject, such as a wound site. The term “subject” is meant the person or organism to which the biocomposite is applied and maintained in contact. As such, subjects may include but are not limited to mammals, e.g., humans and other primates, such as chimpanzees and other apes and monkey species; and the like, as well as non-human subjects such as, but not limited to, birds, mice, rats, dogs, cats, livestock and horses. In certain embodiments, the subject is a human.

In practicing the subject methods, the subject biocomposites containing viable stem cells and platelets may be applied to any convenient internal or external location on the subject, such as to organ tissue including but not limited to integumentary tissue (e.g. sections of the skin), oral tissue (e.g., buccal, tongue, palatal, gums), respiratory tissue (e.g., pharynx, larynx, trachea, bronchi, lungs, diaphragm) gastrointestinal tissue (e.g., esophagus, stomach, liver, gallbladder, pancreas, intestines, colon, rectum and anus.), cardiovascular tissue (e.g., heart, blood vessels), endocrine tissue (e.g., hypothalamus, pituitary gland, pineal body or pineal gland, thyroid, parathyroids, adrenal glands) and genitourinary tissue (kidneys, ureters, bladder, urethra, ovaries, fallopian tubes, uterus, vagina, mammary glands, testes, vas deferens, seminal vesicles, prostate, penis), muscular tissue, nervous tissue (e.g., brain, spinal cord, nerves) as well as soft skeletal tissue (cartilage, ligaments, tendons). Furthermore, the biocomposites may be applied to any type of organismic tissue, including both healthy and diseased tissue (e.g., cancerous, malignant, necrotic, etc.), where desired.

The subject biocomposites may be applied to the body site immediately after preparation (as described above) or may be applied to the body site after a predetermined period of time (i.e., a storage period). For example, the biocomposite may be applied to the body site 30 minutes or more after preparation, such as 60 minutes or more, such as 2 hours or more, such as 6 hours or more, such as 12 hours or more, such as 24 hours or more and including 48 hours or more after preparing the biocomposite.

The size of the body site (e.g., wound site) treated may vary, such as having a surface area ranging from 0.1 to 100 cm², such as 0.5 to 75 cm², such as 1.0 to 50 cm², such as 1.5 to 45 cm², such as 2.0 to 40 cm², such as 2.5 to 35 cm², and including 2 to 30 cm². Where body site treated is three-dimensional cavity, the size of the body site may range from 0.1 to 100 cm³, such as 0.5 to 75 cm³, such as 1.0 to 50 cm³, such as 1.5 to 45 cm³, such as 2.0 to 40 cm³, such as 2.5 to 35 cm³, and including 2 to 30 cm³.

The subject biocomposites may be applied and maintained at the application site over an extended period of time, as desired. For example, the biocomposite may be maintained at the body site (e.g., wound site) over the course of hours, days and including weeks, such as for 6 hours or longer, such as 12 hours or longer, such as 24 hours or longer, such as 48 hours or longer, such as 72 hours or longer, such as after 96 hours or longer, such as 120 hours or longer, such as 144 hours or longer and including 168 hours or longer.

In practicing the subject methods, biocomposites containing viable stem cells and platelets as described herein may be applied a single time or a plurality of times over a given time period, e.g., during the course of wound repair, where the application schedule when a plurality of biocomposites are applied over a given time period may be hourly, daily, weekly, etc. For example, the subject methods include multiple application intervals. By “multiple application intervals” is meant more than one biocomposite is applied and maintained in contact with the subject in a sequential manner. As such, a first biocomposite is removed from contact with the subject and a second biocomposite is reapplied to the subject. In practicing methods of the disclosure, treatment regimens may include two or more application intervals, such as three or more application intervals, such as four or more application intervals, such as five or more application intervals, including ten or more application intervals.

The duration between application intervals in a multiple application interval treatment regimen may vary, as determined by a qualified health care professional. For example, the duration between application intervals in a multiple application treatment regimen may be predetermined and follow at regular intervals. As such, the time between application intervals may vary and may be 1 hour or longer, such as 2 hours or longer, such as 3 hours or longer, such as 6 hours or longer, such as 12 hours or longer, such as 24 hours or longer, such as 48 hours or longer, such as 72 hours or longer, including 168 hours or longer.

In certain instances, a subsequent application interval in a treatment regimen may employ the same or a different formulation of biocomposite as the previous application interval. For example, the concentration of one or more of the viable stem cells or platelets may be increased or decreased in subsequent application intervals or subsequent applied biocomposites may include one or more other components, such as red blood cells, adipose tissue fragments, adipocytes, preadipocytes, fibroblasts, endothelial precursor cells, endothelial cells, macrophages and leukocytes, as described above.

In certain embodiments, the subject methods include assessing a subject as in need of treatment with one or more of the subject biocomposites described above. Individuals may be assessed using any convenient protocol. For example, methods may include determining that a wound site of the subject is susceptible to adhesion formation, such as post-surgical adhesion formation. Diagnosis or assessment of target condition can be performed using any convenient diagnostic protocol as determined by a qualified health care professional.

In certain embodiments, the subject biocomposites can be applied concurrent with other therapeutic protocols, such as for example, for management of blood clotting (e.g., anticoagulation protocols, anti-thrombotic protocols), as well as with devices to physically separate tissues at the body site (e.g., to inhibit adhesion formation). By “concurrent application” is intended administration to a subject such that the therapeutic effect of the combination is caused in the subject undergoing therapy.

In certain embodiments, methods include delivering one or more bioactive agents to the body site with the subject biocomposites. In these embodiments, method may include delivering one or more types of bioactive agents, such as two or more types, such as three or more types, such as four or more types, such as five or more types and including ten or more types of bioactive agents. The amount of bioactive agent delivered may be 0.001 mg or more, such as 0.01 mg or more, such as 0.1 mg or more, such as 0.5 mg or more, such as 1 mg or more and including 1 mg or more. For example, the amount of bioactive agent delivered may range from 0.001 mg to 1000 mg, such as 0.01 mg to 500 mg, such as 0.1 mg to 250 mg, such as 0.5 mg to 100 mg, such as 1 mg to 50 mg, including 1 mg to 10 mg.

As discussed above, example bioactive agents that may be delivered, include but are not limited to, interferon, interleukin, erythropoietin, granulocyte-colony stimulating factor (GCSF), stem cell factor (SCI:), leptin (OB protein), interferon (alpha, beta, gamma), antibiotics such as vancomycin, gentamicin ciprofloxacin, amoxycillin, lactobacillus, cefotaxime, levofloxacin, cefipime, mebendazole, ampicillin, lactobacillus, cloxacillin, norfloxacin, tinidazole, cefpodoxime, proxctil, azithromycin, gatifloxacin, roxithromycin, cephalosporin, anti-thrombogenics, aspirin, ticlopidine, sulfinpyrazone, heparin, warfarin, growth factors, differentiation factors, hepatocyte stimulating factor, plasmacytoma growth factor, glial derived neurotrophic factor (GDNF), neurotrophic factor 3 (NT3), fibroblast growth factor (FGF), transforming growth factor (TGF), platelet transforming growth factor, milk growth factor, endothelial growth factors, endothelial cell-derived growth factors (ECDGF), alpha-endothelial growth factors, beta-endothelial growth factor, neurotrophic growth factor, nerve growth factor (NGF), vascular endothelial growth factor (VEGF), 4-1 BB receptor (4-IBBR), TRAIL (TNF-related apoptosis inducing ligand), artemin (GFRalpha3-RET ligand), BCA-I (B cell-attracting chemokinel), B lymphocyte chemoattractant (BLC), B cell maturation protein (BCMA), brain-derived neurotrophic factor (BDNF), bone growth factor such as osteoprotegerin (OPG), bone-derived growth factor, thrombopoietin, megakaryocyte derived growth factor (MDGF), keratinocyte growth factor (KGF), platelet-derived growth factor (PDGF), ciliary neurotrophic factor (CNTF), neurotrophin 4 (NT4), granulocyte colony-stimulating factor (GCSF), macrophage colony-stimulating factor (mCSF), bone morphogenetic protein 2 (BMP2), BRAK, C-IO, Cardiotrophin 1 (CTI), CCR8, anti-inflammatory: paracetamol, salsalate, diflunisal, mefenamic acid, diclofenac, piroxicam, ketoprofen, dipyrone, acetylsalicylic acid, anti-cancer drugs such as aliteretinoin, altertamine, anastrozole, azathioprine, bicalutarnide, busulfan, capecitabine, carboplatin, cisplatin, cyclophosphamide, cytarabine, doxorubicin, epirubicin, etoposide, exemestane, vincristine, vinorelbine, hormones, thyroid stimulating hormone (TSH), sex hormone binding globulin (SHBG), prolactin, luteotropic hormone (LTH), lactogenic hormone, parathyroid hormone (PTH), melanin concentrating hormone (MCH), luteinizing hormone (LHb), growth hormone (HGH), follicle stimulating hormone (FSHb), haloperidol, indomethacin, doxorubicin, epirubicin, amphotericin B, Taxol, cyclophosphamide, cisplatin, methotrexate, pyrene, amphotericin B, anti-dyskinesia agents, Alzheimer vaccine, antiparkinson agents, ions, edetic acid, nutrients, glucocorticoids, heparin, anticoagulation agents, antivirus agents, anti-HIV agents, polyamine, histamine and derivatives thereof, cystineamine and derivatives thereof, diphenhydramine and derivatives, orphenadrine and derivatives, muscarinic antagonist, phenoxybenzamine and derivatives thereof, protein A, streptavidin, amino acid, beta-galactosidase, methylene blue, protein kinases, beta-amyloid, lipopolysaccharides, eukaryotic initiation factor-4G, tumor necrosis factor (TNF), tumor necrosis factor-binding protein (TNF-bp), interleukin-1 (to 18) receptor antagonist (IL-Ira), granulocyte macrophage colony stimulating factor (GM-CSF), novel erythropoiesis stimulating protein (NESP), thrombopoietin, tissue plasminogen activator (TPA), urokinase, streptokinase, kallikrein, insulin, steroid, acetaminophen, analgesics, antitumor preparations, anti-cancer preparations, anti-proliferative preparations or pro-apoptotic preparations, among other types of bioactive agents.

Depending on the composition of the biocomposite (e.g., presence of adipose tissue, fibrin network, etc.), methods may include delivering the one or more bioactive agents by sustained or pulsatile release. For example, where methods include delivering one or more bioactive agents by “sustained release” a constant and continuous delivery of one or more bioactive agents is maintained while biocomposite is in contact with the site of administration (e.g., abdominal cavity), such as over the course of 1 day or longer, such as 2 days or longer, such as 3 days or longer, such as 5 days or longer and including 7 days or longer. In other instances, methods include delivering one or more bioactive agents by “pulsatile release” such as by releasing one or more bioactive agents into the site of administration incrementally (e.g., at discrete times), such as every 1 hour, such as every 2 hours, such as every 5 hours, such as every 12 hours, such as every 24 hours, such as every 36 hours, such as every 48 hours, such as every 72 hours, such as every 96 hours, such as every 120 hours, such as every 144 hours and including every 168 hours.

Depending on the size of the body site being treated, methods may include delivering an average cumulative amount of bioactive agent of 5 μg/cm² or greater over an extended period of time. In these embodiments, methods include delivering an average cumulative amount of bioactive agent may be 25 μg/cm² or greater, such as 50 μg/cm²or greater, such as 75 μg/cm² or greater over a predetermined delivery interval, such as 100 μg/cm²or greater, such as 125 μg/cm² or greater, such as 150 μg/cm² or greater and including 200 μg/cm² over a predetermined delivery interval.

In yet other embodiments, methods include delivering a target dosage of bioactive agent, such as for example as characterized by total bioactive agent exposure or by average daily bioactive agent exposure. The term target dosage is meant the amount of bioactive agent which is delivered to the subject and may vary depending on the physicochemical properties, mechanical properties, and release rates from the subject biocomposite as well as the site of application. For example, the target dosage of bioactive agent delivered may be 0.01 mg/day or greater, such as 0.04 mg/day or greater, such as 0.5 mg/day or greater over a 4 week dosage interval, such as 1.0 mg/day or greater, such as 2 mg/day or greater, such as 5 mg/day or greater and including 10 mg/day over a 4 week dosage interval.

In some embodiments, methods of the present disclosure include using the subject biocomposites to deliver to a body site of a subject a dosage of about 0.01 mg/kg to 500 mg/kg of the bioactive agent per day, such as from 0.01 mg/kg to 400 mg/kg per day, such as from 0.01 mg/kg to 200 mg/kg per day, such as from 0.1 mg/kg to 100 mg/kg per day, such as from 0.01 mg/kg to 10 mg/kg per day, such as from 0.01 mg/kg to 2 mg/kg per day and including from 0.02 mg/kg to 2 mg/kg per day. In other embodiments, methods include using the subject biocomposites to deliver to a body site of a subject a dosage of from 0.01 to 100 mg/kg four times per day (QID), such as from 0.01 to 50 mg/kg QID, such as from 0.01 mg/kg to 10 mg/kg QID, such as from 0.01 mg/kg to 2 mg/kg QID, such as from 0.01 to 0.2 mg/kg QID. In other embodiments, methods include using the subject biocomposites to deliver to a body site of a subject a dosage of from 0.01 mg/kg to 50 mg/kg three times per day (TID), such as from 0.01 mg/kg to 10 mg/kg TID, such as from 0.01 mg/kg to 2 mg/kg TID, and including from 0.01 mg/kg to 0.2 mg/kg TID. In yet other embodiments, methods include using the subject biocomposites to deliver to a body site of a subject a dosage of from 0.01 mg/kg to 100 mg/kg two times per day (BID), such as from 0.01 mg/kg to 10 mg/kg BID, such as from 0.01 mg/kg to 2 mg/kg BID, including from 0.01 mg/kg to 0.2 mg/kg BID.

### Systems for Preparing Biocomposites Having Viable Stem Cells and Platelets by Centrifugation

Aspects of the present disclosure also include systems for practicing the subject methods. As discussed above, methods for preparing biocomposites having a high concentration of viable stem cells and platelets according to embodiments of the present disclosure include 1) combining a first composition having viable stem cells with a second composition having platelets in a centrifugation vessel; 2) subjecting the centrifugation vessel to a force of centrifugation to produce two or more fractions such that each fraction includes a component having a different density; and 3) collecting a fraction from the centrifuge vessel that includes platelets and viable stem cells. In embodiments, systems are configured to apply a force of centrifugation to the combined first and second composition in a centrifugation vessel for a duration sufficient to fractionate the components of the sample into two or more fractions (e.g., layers), each fraction containing components of different density. A fraction containing a high concentration of viable stem cells and platelets as compared to the concentration of the viable stem cells in the first composition and the concentration of the platelets in the second composition is prepared.

Systems for preparing biocomposites having a high concentration of viable stem cells and platelets include a centrifuge for applying a force of centrifugation to the combined first and second composition. The term “centrifuge” is used herein in its conventional sense to refer to an apparatus for rotating one or more centrifugation vessels containing the first composition and second composition about a rotation axis to apply a centrifugal force to the components in the centrifugation vessel. Any convenient centrifuge protocol may be employed, including but not limited to fixed-angle centrifuges, swinging bucket centrifuges, ultracentrifuges, solid bowl centrifuges, conical centrifuges, among other types of centrifuges. In certain embodiments, the centrifuge is a centrifuge with a horizontal rotor. In other embodiments, the centrifuge is a centrifuge with a fixed angle rotor. For example, the centrifuge may be certain instances a Horizon Model 755VES centrifuge (Drucker Co., Port Matilda Pa.) having a horizontal rotor or fixed angle rotor and brushless DC motor.

As described above, the subject centrifuges may be configured to apply a force of centrifugation which varies, depending on the type of sample, size of centrifugation vessel and desired separation of components. In embodiments, centrifuges of interest may apply a force of centrifugation which ranges (in relative centrifugal force, RCF) from 1 g to 50,000 g, such as from 2 g to 45,000 g, such as from 3 g to 40,000 g, such as from 5 g to 35,000 g, such as from 10 g to 25,000 g, such as from 100 g to 20,000 g, such as from 500 g to 15,000 g and including from 1000 g to 10,000 g. Accordingly, centrifuges of interest may be configured to operate a rotation speeds which vary widely, such as from 1×10³ revolutions per minute (rpm) to 1000×10³ rpm, such as from 2×10³ rpm to 900×10³ rpm, such as from 3×10³ rpm to 800×10³ rpm, such as from 4×10³ rpm to 700×10³ rpm, such as from 5×10³ rpm to 600×10³ rpm, such as from 10×10³ rpm to 500×10³ rpm and including from 25×10³ rpm to 100×10³ rpm.

The centrifuge may also be a temperature controlled centrifuge, where the temperature of the contents in the centrifugation vessel may be maintained or changed (e.g., increased or decreased) as desired. For example, the centrifuge may be configured to maintain the temperature of the sample in the subject devices from −80° C. to 100° C., such as from −75° C. to 75° C., such as from −50° C. to 50° C., such as from −25° C. to 25° C., such as from −10° C. to 10° C., and including from 0° C. to 25° C.

Centrifuges of interest may also be configured with monitoring protocols for assessing the contents of the centrifugation vessel during centrifugation. For example, the centrifuge may include a viewing window to visually observe centrifugation or may include one or more sensors, such as laser scatter sensors, fluorescence sensors, phosphorescence sensors, chemiluminescence sensor, diffuse reflectance sensors, infrared sensors, among other sensing protocols.

In certain embodiments, systems of interest further include computer-controlled systems for practicing the subject methods, where the systems may include one or more computers for automation or semi-automation of a system for practicing methods described herein. In these embodiments, systems may include a computer having a computer readable storage medium with a computer program stored thereon, where the computer program when loaded on the computer includes algorithm for controlling a liquid dispensing device to introduce the first composition (e.g., lipoaspirate or a centrifuged fraction thereof) and the second composition (e.g., whole blood, peripheral blood, bone marrow aspirate, platelet-rich plasma) into the centrifugation vessel, algorithm for subjecting the centrifugation vessel to a force of centrifugation to produce two or more fractions and algorithm for controlling a liquid collection device to collect a fraction containing viable stem cells and platelets. In certain embodiments, the computer program may also include algorithm for providing lipoaspirate or a blood sample from a source to the liquid dispensing device. For example, the computer processor may also include algorithm for transferring lipoaspirate or whole blood from a blood collection tube into the centrifugation vessel.

In embodiments, the computer controlled system includes an input module and a processing module. In some embodiments, the subject systems may include an input module such that parameters or information about: 1) each composition including the type of composition (e.g., lipoaspirate, a centrifuged fraction of lipoaspirate, whole blood, peripheral blood, a blood derivative, citrated blood, platelet-rich plasma, etc.), viscosity of the composition, volume and number of separated fractions expected; 2) components from the sample that are of interest (e.g., viable stem cells (e.g., mesenchymal stromal cells), platelets, adipose tissue fragments, adipocytes, preadipocytes, fibroblasts, endothelial precursor cells, endothelial cells, macrophages, leukocytes and red blood cells; 3) desired speed of the centrifuge for applying the force of centrifugation; 4) the temperature of the centrifuge and 5) the number of centrifugation intervals, etc. may be inputted into the computer. The processing module includes memory having a plurality of instructions for performing certain steps of the subject methods, such as introducing the first and second compositions into the centrifugation vessel, applying a force of centrifugation as well as instructions for collecting the desired fractions.

The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

Computer systems of interest may include a display and operator input device. Operator input devices may, for example, be a keyboard, mouse, or the like. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described comprising a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid state storage devices (including magnetic or optical disks or tape or RAM, or any other suitable device, either fixed or portable). The processor may include a general purpose digital microprocessor suitably programmed from a computer readable medium carrying necessary program code. Programming can be provided remotely to the processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (e.g., smartphone) or tablet device.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g, over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a work station, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows NT®, Windows XP, Windows 7, Windows 8, iOS, Sun Solaris, Linux, OS/400, Compaq Tru64 Unix, SGI IRIX, Siemens Reliant Unix, and others.

### Kits

Aspects of the invention further include kits, where kits include one or more components for preparing and using the subject biocomposites containing viable stem cells and platelets. In certain embodiments, kits include one or more devices for preparing (e.g., harvesting) the components of the biocomposite, such as lipoaspirate, whole blood, peripheral blood, bone marrow aspirate or platelet rich plasma. In some embodiments, kits include a device for preparing the a composition that includes viable stem cells from lipoaspirate from a subject. In certain instances, the composition that includes viable stem cells is prepared from lipoaspirate from a subject using a collection and centrifugation container such as described in International Patent Application No. PCT/US2013/000036 published as WO2013/122683 on Aug. 22, 2013 as well as U.S. Provisional Patent Application No. 62/002,052 filed on May 22, 2014, the disclosures of which are herein incorporated by reference.

In other embodiments, kits include a blood collection device for obtaining a whole blood sample. In yet other embodiments, kits include a plasma preparation device for obtaining platelet-rich plasma. In certain instances, kits include a source of fibrinogen and plasminogen, such as prepared using a centrifugation container as described in co-pending U.S. patent application Ser. No. 13/199,129 filed on Aug. 19, 2011, U.S. patent application Ser. No. 13/199,111 filed on Aug. 19, 2011, U.S. patent application Ser. No. 13/199,119 filed on Aug. 19, 2011 as well as U.S. Provisional Patent Application No. 62/069,783 filed on Oct. 28, 2014, the disclosures of which are herein incorporated by reference. In certain embodiments, kits include one or more devices for preparing a source of thrombin. In some instances, the subject kits may include devices for preparing thrombin such as those described in International Patent Application No. PCT/US2013/061756 published as WO2014/052496 on Apr. 3, 2014, the disclosure of which is herein incorporated by reference.

In certain embodiments, kits include a three-dimensional mold for combining the subject biocomposites containing viable stem cells with one or more of fibrinogen, thrombin, plasminogen, fibrin gel and adipose tissue. As discussed above, the three-dimensional mold may be any suitable container and may be in the shape of a circle, oval, half-circle, crescent-shaped, star-shaped, square, triangle, rhomboid, pentagon, hexagon, heptagon, octagon, rectangle or other suitable polygon or may be a tube, such as test tube, a centrifuge tube, conical bottom tube as well as tubing. For example, the three-dimensional mold may be a container having the shape of a cube, cone, half sphere, star, triangular prism, rectangular prism, hexagonal prism or other suitable polyhedron or may be in the shape of a tube. The mold may include one or more ports for inputting the biocomposite containing viable stem cells as well as the source of thrombin, fibrinogen, plasminogen or adipose tissue. Any suitable port configuration may be employed, where examples of ports include channels, orifices, channels having a check valve, a Luer taper fitting, a port with a breakable seal (e.g., single use ports) among other types of ports. In some embodiments, the port is configured to connect to a syringe. In other embodiments, the port is configured to facilitate access for a needle into the cavity of the container to aspirate, mix and remove components from the container. In certain embodiments, the port is configured with a Luer taper fitting, such as a Luer-Lok or a Luer-slip. In some embodiments, kits include one or more syringes for inputting components into the mold. Where desired, syringes may be configured with a Luer taper fitting, such as a Luer-Lok or a Luer-slip for connection to the three-dimensional mold or may be configured with a conduit (e.g., tubing) to fluidly connect one or more syringes to three-dimensional mold. Syringes, as well as conduits when present, may also include one or more valves such as a stop-cock valve for controlling the rate of inputting into the three-dimensional mold.

In some instances, the kits can include one or more additional components (e.g., buffers, water, solvent etc.). In some instances, the kits may further include a sample collection device, e.g., blood collection device such as an evacuated blood collection tube, needle, syringe, pipette, tourniquet, etc. as desired.

The various components of the kits may be present in separate containers, or some or all of them may be pre-combined. For example, in some instances, one or more components of the kit, e.g., the three-dimensional mold, syringes, lipoaspirate preparation device, platelet-rich plasma preparation container and thrombin preparation device are present in a sealed pouch, e.g., a sterile foil pouch or envelope.

In addition to the above components, in certain instances the subject kits may further include instructions for assembling the subject kit components as well as for practicing methods for preparing the biocomposites containing viable stem cells and platelet rich plasma as described herein. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

### Utility

The subject biocomposites containing a high concentration of viable stem cells and platelets and methods find use in a variety of applications during wound repair from trauma or surgery or in the treatment or prevention of fistulas, skin excisions, skin ulcers and burns. In certain embodiments, the present disclosure finds use in preventing adhesion formation at a wound site during tissue repair at a body site. In some embodiments, the present disclosure provides for wound healing while promoting one or more of hemostasis, reduced time for wound closure, reduced post-surgical wound complications, reduced scarring as we as enhanced cosmetic appearance of the wound subsequent to healing. Embodiments also find use where a subject would benefit from delivery of an active agent. Likewise, biocomposites of interest also find use in any application where a bioactive agent would benefit from a tunable biocompatible and biodegradable delivery vehicle which could be used to stabilize or provide site specific delivery of the bioactive agent.

In certain examples, biocomposites having a high concentration of viable stem cells find use during the repair of tissue at a wound site. In another example, the subject biocomposites find use in delivery of growth factors (e.g., tissue growth factors), hemostatics, pharmaceuticals or other active agents used to treat and ailment where delivery to a site of administration can be made using a biocompatible delivery vehicle such as the biocomposites described herein. As described above, treatment is meant that at least an amelioration of the symptoms associated with the condition afflicting the subject is achieved, where amelioration is used in a broad sense to refer to at least a reduction in the magnitude of a parameter, e.g., symptom, associated with the condition being treated. As such, treatment also includes situations where the pathological condition, or at least symptoms associated therewith, are completely inhibited, e.g., prevented from happening, or stopped, e.g., terminated, such that the subject no longer suffers from the condition, or at least the symptoms that characterize the condition.

Although the foregoing invention has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method comprising:
combining a first composition comprising viable stem cells with a second composition comprising platelets in a centrifugation vessel;
subjecting the centrifugation vessel to a force of centrifugation to produce two or more fractions, wherein each fraction comprises a component having a different density;
collecting a fraction from the centrifuge vessel that comprises the platelets and viable stem cells.

2-3. (canceled)

4. The method according to claim 1, wherein the second composition is selected from the group consisting of whole blood, peripheral blood and bone marrow aspirate.

5. The method according to claim 4, wherein the second composition is whole blood.

6. The method according to claim 5, wherein the whole blood comprises an anti-coagulant.

7. The method according to claim 4, wherein the second composition comprises a centrifuged fraction of whole blood comprising platelets and red blood cells.

8. The method according to claim 1, wherein the second composition is platelet rich plasma.

9. The method according to claim 1, wherein the first composition and the second composition are from the same subject.

10. The method according to claim 1, wherein the centrifugation vessel is subjected to the centrifugal force of from 100 g to 10,000 g.

11. (canceled)

12. The method according to claim 1, wherein the centrifugation vessel is subjected to the centrifugal force for a duration of from 1 minute to 120 minutes.

13-15. (canceled)

16. The method according to claim 1, wherein the collected fraction comprises a volume that is 30% or less than the total volume of the first and second composition.

17. (canceled)

18. The method according to claim 1, wherein the collected fraction comprises adipose tissue fragments.

19. The method according to claim 1, further comprising performing lipoplasty on a subject to harvest lipoaspirate from the subject.

20. (canceled)

21. The method according to claim 19, further comprising harvesting the adipose tissue component from the lipoaspirate.

22. A method for the preparation of a cell composition suitable for achieving a therapeutic or cosmetic effect when applied to a body comprising the following steps:
combining a first fluid fraction comprising adipose tissue derived mesenchymal stromal cells and red blood cells with a second fluid fraction comprising platelets and red blood cells to create a first cell mixture having a first concentration of mesenchymal stromal cells and a first concentration of platelets and a first total number of red blood cells;
centrifuging said first cell mixture in a centrifuge vessel for sufficient time and gee force to stratify the fluid mixture to create a low density fraction substantially depleted of said mesenchymal stromal cells, platelets and red blood cells; an intermediate density phase fluid fraction that is enriched in concentration of said platelets and said mesenchymal stromal cells and a high density fluid fraction that is enriched for red blood cells and substantially depleted of said mesenchymal stromal cells and said platelets;
harvesting at least a fraction of said intermediate density phase fluid containing the majority of mesenchymal stromal cells and platelets from said centrifuge vessel to create a second cell mixture wherein both the concentration of platelets and mesenchymal stromal cells in said second cell mixture is at least greater by 1.6 fold than the concentration of platelets and mesenchymal stromal cells in said first cell mixture and the total number of red blood cells in said second cell mixture is less than half of total red blood cells in said first cell mixture.

23. The method according to claim 22, wherein all of the cells in the first mixture are derived from the same patient.

24-34. (canceled)

35. A liquid composition comprising viable stem cells and platelets,
wherein the concentration of the stem cells in the composition is 1000 cells/mL or more and the concentration of platelets in the composition is 1×10⁶ platelets/μL or more.

36. The composition according to claim 35, further comprising adipose tissue fragments.

37-38. (canceled)

39. The composition according to claim 35, further comprising one or more of fibrin, fibrinogen, plasmin, plasminogen and thrombin.

40-42. (canceled)

43. The composition according to claim 35, further comprising a bioactive agent.

44. (canceled)

45. The composition according to claim 35, further comprising a bone graft.

46-47. (canceled)

48. The method according to claim 1, further comprising contacting a body site with the collected fraction, wherein the collection fraction comprises viable stem cells and platelets, wherein the concentration of the stem cells in the composition is 1000 cells/mL or more and the concentration of platelets in the composition is 1×10⁶ platelets/μL or more.

49-78. (canceled)
